(19) United States
(12) Patent Application Publication
(10) Pub. No.: US 2006/0084176 A1
(43) Pub. Date: Apr. 20, 2006

(54) Detection of uronium salts

(75) Inventor: Joseph Almog, Mevasseret-Zion (IL)

Correspondence Address: Martin Moynihan;c/o Anthony Castorina, Suite 207, 2001 Jefferson Davis Highway, Arlington, VA, 22202, US

(73) Assignee: Yissum Research Development Company of the Hebrew University of Jerusalem, Jerusalem (IL)

(21) Appl. No.: 11/075,685

(22) Filed: Mar. 10, 2005

Related U.S. Application Data
(60) Provisional application No. 60/558,104, filed on Apr. 1, 2004.

(30) Foreign Application Priority Data
Apr. 1, 2004 (IL) 161219

(51) Int. Cl. G01N 33/00

(52) U.S. Cl. 436/110

(57) ABSTRACT

Methods and kits for colorimetric identification of uronium salts, such as explosives.

### RELATIONSHIP TO EXISTING APPLICATIONS

This application claims priority from U.S. Provisional Patent Application No. 60/558,104, filed Apr. 1, 2004, and Israel Patent Application No. 161219, also filed Apr. 1, 2004, the contents of which are hereby incorporated by reference.

### FIELD AND BACKGROUND OF THE INVENTION

The present invention relates to the field of colorimetry, and especially to colorimetric identification of uronium salts such as urea nitrate.

Urea nitrate is a crystalline acid salt of urea often used as a fertilizer. There is evidence that in the urea nitrate crystal the urea oxygen is protonated. Pure urea nitrate is a white crystalline powder that is visually indistinguishable from sugar.

Urea nitrate is explosive and is therefore frequently used as a component of high explosive charges. Militants are known to favor the use of urea nitrate due to the fact that even unskilled workers can easily prepare large amounts of this relatively stable explosive from commonly available components without sophisticated laboratory equipment. Terrorists acting in Israel often use urea nitrate to attack civilian targets. Notoriously, terrorists used a urea nitrate-containing explosive device in an attack on the New York World Trade Center in February 1993. The difficulty of identifying urea nitrate is illustrated by allegations surrounding the investigation of this attack on the World Trade Center. Senior FBI investigator F. W. Whitehurst allegedly demonstrated that using prior art methods it is difficult to differentiate between urea nitrate remnants and urine spilled from sewage pipes destroyed in the explosion.

It is generally useful to have a method to allow identification of a suspicious white powder as urea nitrate. Preferably such a test should be simple and accurate, allowing a person who is under the extreme pressure of handling a potential explosive to accurately and quickly provide a definitive answer if a sample is potentially urea nitrate or certainly not urea nitrate.

It is known that when it is necessary to quickly identify the presence of a certain compound or type of compound in a sample, a preferred method is colorimetry.

In colorimetry, a sample to be identified is contacted with a reagent or series of reagents, including a chromogenic indicator. A color change by the indicator indicates the presence or absence of a threshold concentration of a compound or type of compound in a sample. One of the greatest advantages of colorimetric methods is robustness. Even physically and mentally exhausted less-skilled operators in non-laboratory situations under time pressure can quickly identify suspect compounds to make a quick and accurate decision. It is important to note, however, that the exact nature of a compound identified by a colorimetric test is determined by the context in which the test is performed.

Many different compositions containing chromogenic reagents used in colorimetry are known: litmus paper for the detection of H⁺ concentrations; Benedict's solution for the detection of monosaccharide sugars; Lugol's solution for the detection for the detection of starch; bromothymol blue for the detection of carbon dioxide; a methanolic solution of cobaltous thiocyanate, ammonium metavanidate and yellow dye for the detection of heroin; and 4-(4-nitrobenzyl)pyridine for the detection of 1,1-thiobis(2-chloroethane).

Many methods for identifying amine-containing compounds based on the use of an acidic solution of an aromatic aldehyde as a chromogenic indicator are known.

Ehrlich's reagent is an aqueous solution of 2% p-dimethylaminobenzaldehyde (p-DMAB) with 20% HCl used, for example, for the detection of urobilin in urine, hallucinogens (e.g. psilocybin, 5-MEO DMT, LSD), sulfonamides and indoles. Variants of Ehrlich's reagent using other acids and solvents are known (e.g. acetone/water solvent or glacial acetic acid/perchloric acid).

Kovac's reagent is an isoamylalcohol solution of p-DMAC with H₂SO₄ used, for example, for the detection of indoles.

Van Urk's reagent is an acid aqueous solution of p-dimethylaminobenzaldehyde (p-DMAB) with H₂SO₄ used, for example, for the detection of indoles.

Renz and Loew's reagent is an isoamylalcohol solution of p-DMAC with HCl used, for example, for the detection of indoles.

Other instances where highly acidic solutions of p-DMAC are used as indicators are described in U.S. Pat. No. 4,395,494, U.S. Pat. No. 4,277,248, U.S. Pat. No. 5,366,872 and U.S. Pat. No. 5,741,659.

In all these reactions, the pH of the reagent solution is lower than about 2. Such a low pH is necessary for the chromogenic reaction to take place. It is generally accepted that the reaction of an aromatic aldehyde with an amine involves a nucleophilic attack by the free-electron pair of the amine on the carbon of the oxygen protonated aldehyde followed by loss of water to yield an imine. This process is depicted for p-DMAC.

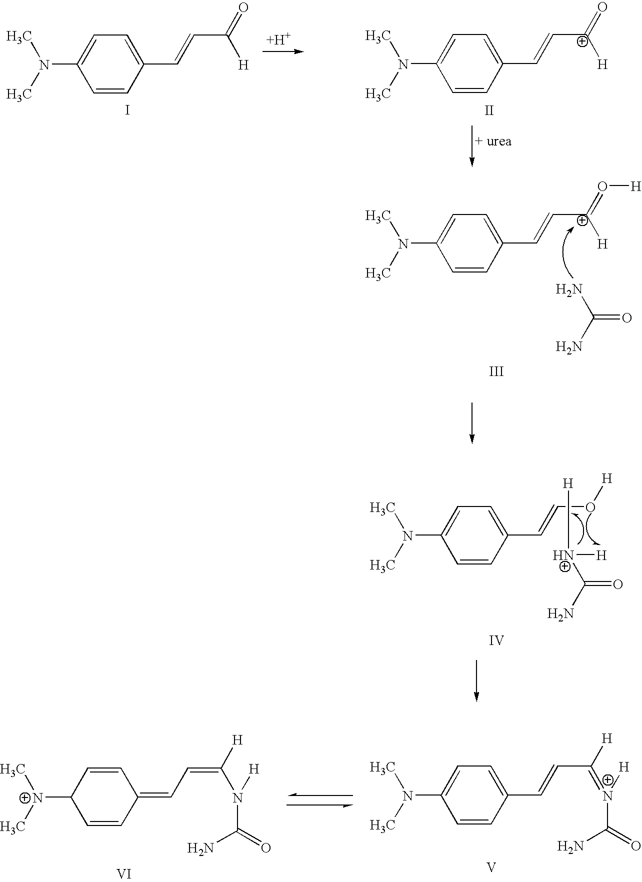

p-DMAC (I) is protonated at the aldehyde oxygen due to the highly acid conditions in a reagent solution, forming a positive charge on the aldehyde carbon (II). The free-electron pair of a nitrogen atom in a primary or secondary amine-containing sample molecule makes a nucleophilic attack on the positively charged carbon atom (III), followed by the loss of water (IV) to make an imine (V). Due to the positive charge stabilizing properties of the p-dimethylaminocinnamic group, the intensely red colored dialkylaminium ion (VI) is significantly more abundant than the imine (V), indicating the presence of an amine-containing molecule.

There are a number of disadvantages in using the prior art methods using acidic aromatic aldehyde solutions for the colorimetric identification of urea nitrate.

First, the solutions are all very acidic, having a pH of less than 2. A solution having a pH of 2 or less is an irritant and preferably not used for large scale screening due to health concerns.

Second, due to the ubiquity of amine-containing interferants, a positive chromogenic reaction would often be a false positive. A prior art aromatic aldehyde reagent solution indicates that a sample contains an amine and does not differentiate between urea nitrate and likely amine-containing interferants such as sewage, urea or LSD.

There is, hence, a widely recognized need for, and it would be highly advantageous to have a simple and efficient method to identify urea nitrate in a sample. It is most preferable that such a method be colorimetric and be implemented as an easy to use kit.

### SUMMARY OF THE INVENTION

The present invention provides sensitive and simple colorimetric methods and kits for the identification of uronium salts, e.g. urea nitrate. The present invention is based on the use of an indicator that reacts in the presence of an uronium salt but not in the presence of non-acidified amines. This is achieved by providing the indicator in a solution that is not acidic enough to allow the formation of the chromophore. When a uronium salt is examined, the solution is acidified by the uronium salt itself and the reaction proceeds.

As used herein, the term “uronium salt” includes a cation of urea or a cation of a urea derivative. Generally intended is a solid or crystalline substance made of a cationized ureic moiety associated with an anion, especially a very weakly basic anion including but not limited to F⁻, Cl⁻, Br⁻, I⁻, ClO₄⁻, SO₄²⁻, NO₃⁻ and PO₄⁻. Included in this definition are salts where the urea or the urea derivative is cationized by a cationizing group R⁺ (R, mutandis mutandi, as defined below) where R⁺ includes, amongst others, H⁺, alkyl⁺ and aryl⁺ cations. It is important to note that as used herein, “uronium salt” implies no structural limitations, e.g. as concerns the location of the cation relative to the ureic moiety. For example, included in the definition of “uronium salt” are urea or urea derivatives where the cationizing entity is associated with the ureic carbonyl oxygen but also where the cationizing entity is associated with one of the two ureic nitrogens.

As used herein the term “urea derivative” refers to a chemical compound having two nitrogens bound to a carbonyl moiety.

Therefore, according to the teachings of the present invention there is provided the colorimetric identification of uronium salts (e.g. urea nitrate). The colorimetric identification of the present invention differentiates between an uronium salt and common interferants such as urea or LSD.

According to the teachings of the present invention there is also provided for the use of an indicator for determining the presence or absence of an uronium salt (e.g. urea nitrate) in a sample, the indicator selected from the group consisting of an aldehyde and a compound configured to form an aromatic disubstituted aminium ion upon reaction with urea.

According to the teachings of the present invention, there is also provided for the use of an indicator solution for determining the presence or absence of an uronium salt (e.g. urea nitrate) in a sample, the indicator solution having a pH of greater than about 2 and including at least one indicator selected from the group consisting of an aldehyde and a compound configured to form an aromatic disubstituted aminium ion upon reaction with urea. According to a feature of the present invention, the indicator solution has a pH greater than about 3, greater than about 4, greater than about 5, greater than about 6 and even greater than about 7.

An aldehyde used in implementing the teachings of the present invention is preferably an aromatic aldehyde. An aromatic disubstituted aminium ion used in implementing the teachings of the present invention is preferably an aromatic dialkylaminium ion. Especially preferred indicators for implementing the teachings of the present invention include, but are not limited to, p-dimethylaminocinnamaldehyde (p-DMAC), diethylaminocinnamaldehyde (p-DEAC), p-dimethylaminobenzaldehyde (p-DMAB) or p-diethylaminobenzaldehyde (p-DEAB) and mixtures thereof.

According to the teachings of the present invention there is also provided a method of determining the presence of an uronium salt in a sample comprising forming an aromatic disubstituted aminium ion from an indicator solution, the indicator solution having a pH of greater than about 2, the disubstituted aminium ion of the structure: 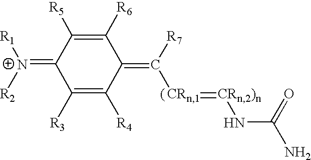
wherein n is 0 or greater; and
wherein R₁, R₂, R₃, R₄, R₅, R₆, R₇, R_{n,1} and R_{n,2} are each independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl, cyano, nitro, amino and —NR₈R₉, R₈ and R₉ independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl and cyano, or alternatively, at least two of R₁, R₂, R₃, R₄, R₅, R₆, R₇, R₈, R₉, R_{n,1} and R_{n,2} are part of an aromatic, heteroaromatic, alicyclic or heteroalicyclic ring. According to a feature of the present invention, the indicator solution has a pH greater than about 3, greater than about 4, greater than about 5, greater than about 6 and even greater than about 7.

According to a feature of the present invention, the aromatic disubstituted aminium ion is an aromatic dialkylaminium ion, R₁ and R₂ each being independently selected from the group consisting of methyl and alkyl.

According to the teachings of the present invention there is provided a method of identifying a sample as containing an uronium salt (e.g. urea nitrate), comprising reacting the sample with an indicator solution including at least one indicator of the structure: 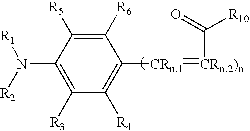
wherein n is 0 or greater; and
wherein R₁, R₂, R₃, R₄, R₅, R₆, R₁₀, R_{n,1} and R_{n,2} are each independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl, cyano, nitro, amino and —NR₈R₉, R₈ and R₉ independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl and cyano, or alternatively, at least two of R₁, R₂, R₃, R₄, R₅, R₆, R₈, R₉, R₁₀, R_{n,1} and R_{n,2} are part of an aromatic, heteroaromatic, alicyclic or heteroalicyclic ring; the indicator solution having a pH greater than about 2. According to a feature of the present invention, the indicator solution has a pH greater than about 3, greater than about 4, greater than about 5, greater than about 6 and even greater than about 7.

According to a preferred embodiment of the present invention, the indicator is selected from the group consisting of p-dialkylaminocinnamaldehydes, p-dialkylaminocinnamyl ketones, linear p-dialkylaminocinnamic acids, cyclic p-dialkylaminocinnamic acids (coumarins), linear p-dialkylaminocinnamamides, cyclic p-dialkylaminocinnamamides (quinolinones), p-dialkylaminobenzaldehydes, p-dialkylaminophenyl ketones, p-dialkylaminobenzoic acids, p-dialkylamino benzamides, salts thereof and esters thereof.

In an embodiment of the present invention, the indicator is a p-dialkylaminocinnamaldehyde of the structure: 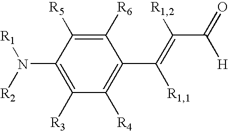
wherein R₁ and R₂ are each independently selected from the group consisting of methyl and alkyl or alternatively, at least one of R₁ or R₂ is part of an aromatic, heteroaromatic, alicyclic or heteroalicyclic ring. Suitable p-dialkylaminocinnamaldehydes include but are not limited to p-dimethylaminocinnamaldehyde (p-DMAC) and p-diethylaminocinnamaldehyde (p-DEAC).

In another embodiment of the present invention, the indicator is a p-dialkylaminocinnamyl ketone of the structure: 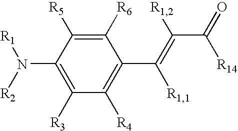
wherein R₁ and R₂ are each independently selected from the group consisting of methyl and alkyl or alternatively, at least one of R₁ or R₂ is part of an aromatic, heteroaromatic, alicyclic or heteroalicyclic ring and where R₁₄ is independently selected from the group consisting of methyl, alkyl, hydroxyalkyl, trihaloalkyl, cycloalkyl, alkenyl, alkynyl, aryl, benzyl, heteroaryl, and heteroalicyclic. Suitable p-dialkylaminocinnamyl ketones include but are not limited to (3E)-4-[4-(dimethylamino) phenyl]-3-buten-2-one, (1E)-1-[4-(dimethylamino) phenyl]-4,4-dimethyl-1-penten-3-one, (2E)-3-[4-(dimethylamino) phenyl]-1-(2-furyl)-2-propen-1-one, (2E)-3-[4-(dimethylamino)phenyl]-1-phenyl-2-propen-1-one, 3-[4-(dimethylamino)phenyl]-1-(4-methylphenyl)-2-propen-1-one, (3E)-4-[4-(diethylamino)phenyl]-3-buten-2-one, 1E)-1-[4-(diethylamino)phenyl]-4,4-dimethyl-1-penten-3-one, (2E)-3-[4-(diethylamino)phenyl]-1-(2-furyl)-2-propen-1-one, (2E)-3-[4-(diethylamino)phenyl]-1-phenyl-2-propen-1-one and 3-[4-(diethylamino)phenyl]-1-(4-methylphenyl)-2-propen-1-one.

In another embodiment of the present invention, the indicator is a linear p-dialkylaminocinnamic acid of the structure: 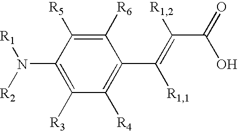
a salt thereof or an ester thereof,
wherein R₁ and R₂ are each independently selected from the group consisting of methyl and alkyl or alternatively, at least one of R₁ or R₂ is part of an aromatic, heteroaromatic, alicyclic or heteroalicyclic ring. Suitable linear p-dialkylaminocinnamic acids include but are not limited to 3-(4-amino-2-methylphenyl) acrylic acid, 4-(dimethylamino) cinnamic acid (CAS 1552-96-1), (2E)-3-[4-(dimethylamino) phenyl]-2-methyl-2-propenoic acid, (2E)-2-cyano-3-[4-(dimethylamino)phenyl]-2-propenoic acid and ethyl 2-cyano-3-[4-(dimethylamino)phenyl] acrylate, salts thereof or esters thereof.

In another embodiment of the present invention, the indicator is a cyclic p-dialkylaminocinnamic acid (coumarin) of the structure: 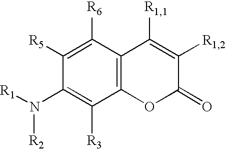
wherein R₁ and R₂ are each independently selected from the group consisting of methyl and alkyl or alternatively, at least one of R₁ or R₂ is part of an aromatic, heteroaromatic, alicyclic or heteroalicyclic ring. Suitable cyclic p-dialkylaminocinnamic acids (coumarins) include but are not limited to coumarin 110 (CAS 20571-42-0), coumarin 6H (CAS 58336-35-9), 3-acetyl-7-(diethylamino)-2H-chromen-2-one, 7-amino-4-methylcoumarin, 7-(diethylamino)coumarin-3,4-dicarboxylic acid (CAS 75240-77-6), 1-[7-(diethylamino)-3-coumarinylcarbonyl]imidazole (CAS 261943-47-9), N-succinimidyl 7-(diethylamino)coumarin-3-carboxylate (CAS 139346-57-9), 7-(diethylamino)coumarin-3-carboxylic acid (CAS 50995-74-9), 7-(diethylamino)coumarin-3-carbonyl azide (CAS 157673-16-0), 7-(diethylamino)coumarin-3-carbohydrazide (CAS 100343-98-4), 7-(diethylamino)coumarin (CAS 20571-42-0), 3-(2-N-methylbenzimidazolyl)-7-N,N-diethylaminocoumarin (CAS 41044-12-6), 3-(2-benzothiazolyl)-7-(diethylamino)coumarin (CAS 38215-36-0), N-succinimidyl 3-(2-benzothiazolyl)-7-(diethylamino)coumarin-4-carboxylate (CAS none) and 3-(2-benzothiazolyl)-7-(diethylamino)coumarin-4-carboxylic acid (CAS 136997-14-3).

In another embodiment of the present invention, the indicator is a cyclic p-dialkylaminocinnamide (quinolinone) of the structure: 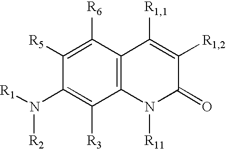
wherein R₁ and R₂ are each independently selected from the group consisting of methyl and alkyl or alternatively, at least one of R₁ or R₂ is part of an aromatic, heteroaromatic, alicyclic or heteroalicyclic ring; and wherein R₁₁ is selected from the group consisting of H, methyl, alkyl, hydroxyalkyl, trihaloalkyl, cycloalkyl, alkenyl, alkynyl, aryl, benzyl, heteroaryl, heteroalicyclic and halo. Suitable cyclic p-dialkylaminocinnamamides (quinolinones) include but are not limited to 7-(dimethylamino)-4-methyl-2(1H)-quinolinone (CAS 26078-23-9) and 7-(dimethylamino)-2(1H)-quinolinone.

In another embodiment of the present invention, the indicator is a linear p-dialkylaminocinnamamide of the structure: 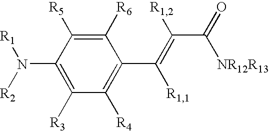
wherein R₁ and R₂ are each independently selected from the group consisting of methyl and alkyl or alternatively, at least one of R₁ or R₂ is part of an aromatic, heteroaromatic, alicyclic or heteroalicyclic ring; and wherein R₁₂ and R₁₃ are each independently selected from the group consisting of H, methyl, alkyl, hydroxyalkyl, trihaloalkyl, cycloalkyl, alkenyl, alkynyl, aryl, benzyl, heteroaryl, heteroalicyclic and halo. Suitable linear p-dialkylaminocinnamamides include but are not limited to 2-cyano-3-[4-(dimethylamino)phenyl]acrylamide and (2E)-2-cyano-3-[4-(diethylamino)phenyl]-N-methyl-2-propenamide.

In another embodiment of the present invention, the indicator is a p-dialkylaminobenzaldehyde of the structure: 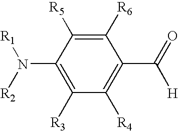
wherein R₁ and R₂ are each independently selected from the group consisting of methyl and alkyl or alternatively, at least one of R₁ or R₂ is part of an aromatic, heteroaromatic, alicyclic or heteroalicyclic ring. Suitable p-dialkylaminobenzaldehydes include but are not limited to p-dimethylaminobenzaldehyde (p-DMAB) and p-diethylaminobenzaldehyde (p-DEAB).

In another embodiment of the present invention, the indicator is a p-dialkylaminophenyl ketone of the structure: 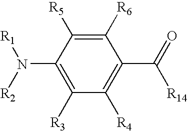
wherein R₁ and R₂ are each independently selected from the group consisting of methyl and alkyl or alternatively, at least one of R₁ or R₂ is part of an aromatic, heteroaromatic, alicyclic or heteroalicyclic ring and where R₁₄ is independently selected from the group consisting of methyl, alkyl, hydroxyalkyl, trihaloalkyl, cycloalkyl, alkenyl, alkynyl, aryl, benzyl, heteroaryl and heteroalicyclic. Suitable p-dialkylaminophenyl ketones include but are not limited to p-dimethylaminophenyl methyl ketone, p-dimethylaminophenyl ethyl ketone, p-dimethylaminophenyl phenyl ketone, p-diethylaminophenyl methyl ketone, p-diethylaminophenyl ethyl ketone, p-diethylaminophenyl phenyl ketone, p-dimethylamino acetophenone 4′-piperidinoacetophenone (CAS 10342-85-5), 4′-piperazinoacetophenone (CAS 51639-48-6), 4′-morpholinoacetophenone (CAS 39910-98-0), 4′-(dimethylamino)-2,2,2-trifluoroacetophenone (CAS 2396-05-6), 1-[4-(4-hydroxy-1-piperidinyl)phenyl] ethanone, 1-[4-(4-morpholinyl)phenyl]-1-propanone and 4-(dimethylamino) phenyl](phenyl) methanone (CAS 530-44-9).

In another embodiment of the present invention, the indicator is a p-dialkylaminobenzoic acid, of the structure: 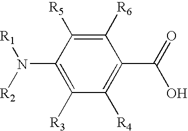
a salt thereof or an ester thereof,
wherein R₁ and R₂ are each are each independently selected from the group consisting of methyl and alkyl or alternatively, at least one of R₁ or R₂ is part of an aromatic, heteroaromatic, alicyclic or heteroalicyclic ring. Suitable p-dialkylaminobenzoic acids include but are not limited to methyl 4-(diphenylamino) benzoate, 4-(dimethylamino) benzoic acid (CAS 619-84-1), 4-(diethylamino)benzoic acid (CAS 5429-28-7), salts thereof or esters thereof.

In another embodiment of the present invention, the indicator is a p-dialkylaminobenzamide of the structure: 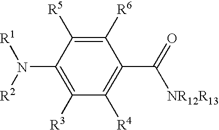
wherein R₁ and R₂ are each independently selected from the group consisting of methyl and alkyl or alternatively, at least one of R₁ or R₂ is part of an aromatic, heteroaromatic, alicyclic or heteroalicyclic ring; and wherein R₁₂ and R₁₃ are each independently selected from the group consisting of H, methyl, alkyl, hydroxyalkyl, trihaloalkyl, cycloalkyl, alkenyl, alkynyl, aryl, benzyl, heteroaryl, heteroalicyclic and halo. Suitable p-dialkylaminobenzamides include but are not limited to p-dimethylaminobenzamide and p-diethylaminobenzamide.

According to the teachings of the present invention there is also provided a method for determining the presence or absence of an uronium salt (e.g. urea nitrate) in a sample comprising contacting the sample with an indicator in an indicator solution having a pH greater than about 2, the indicator selected from the group consisting of an aldehyde and a compound configured to form an aromatic dialkylaminium ion upon reaction with the uronium salt. An appropriate change in the appearance of the indicator solution subsequent to the contacting with the sample indicates the presence of an uronium salt. Appropriate changes in the appearance of the indicator solution include a change in color, a change in light absorption and a change in fluorescence of the solution. According to a feature of the present invention, the indicator solution has a pH greater than about 3, greater than about 4, greater than about 5, greater than about 6 and even greater than about 7. According to a feature of the present invention, a portion of the solution is allowed to evaporate subsequent to the contacting so as to increase the concentration of the sample in the indicator solution.

According to a feature of the present invention, the indicator solution includes at least one protic solvent, that is, a solvent capable of donating protons. Suitable protic solvents include but are not limited to water, alcohol, ethanol, methanol, propanol, isopropanol, butanol, isoamylalcohol, glycol and 1,2-dihydroxypropane.

According to a feature of the present invention, the indicator solution includes, in addition to a first protic solvent, a second solvent, the second solvent being more volatile than the first protic solvent. Preferably the volatility of the second solvent is such that it evaporates when the indicator solution and the sample are contacted, so as to increase the concentration of the sample in the indicator solution. Suitable second solvents include volatile solvents including but not limited to ethanol, methanol, butyl acetate, acetonitrile, chloroform, dichloromethane, diethyl ether and ethyl acetate. In such a case, a preferred first protic solvent is water.

According to a feature of the present invention, the indicator solution includes in addition to at least one protic solvent at least one aprotic solvent. Suitable aprotic solvents include but are not limited to ethers, acetates, ketones, acetone, butyl acetate, acetonitrile, chloroform, dichloromethane, diethyl ether, dimethyl formamide, dimethylsulfoxide, ethyl acetate and tetrahydrofuran. In one preferred embodiment, the solvent is a mixture of ethanol and water.

According to a feature of the present invention, the indicator is selected from the group consisting of p-dialkylaminocinnamaldehydes, p-dialkylaminocinnamyl ketones, linear p-dialkylaminocinnamic acids, cyclic p-dialkylaminocinnamic acids (coumarins), linear p-dialkylaminocinnamamides, cyclic p-dialkylaminocinnamamides (quinolinones), p-dialkylaminobenzaldehydes, p-dialkylaminophenyl ketones, p-dialkylaminobenzoic acids, p-dialkylamino benzamides, salts thereof and esters thereof.

According to a feature of the present invention, the indicator is an aldehyde of the structure R—CHO wherein R is selected from the group consisting of alkyl, hydroxyalkyl, trihaloalkyl, cycloalkyl, alkenyl, alkynyl, aryl, benzyl, heteroaryl, heteroalicyclic, aminobenzyl, alkylaminobenzyl, dialkylaminobenzyl, dialkoxyaminobenzyl, cinnamyl, aminocinnamyl, alkyaminocinnamyl, dialkyaminocinnamyl, alkoxyaminocinnamyl, dialkoxyaminocinnamyl, nitrobenzyl, alkylbenzyl, alkoxybenzyl, phenol, dihydroxyphenyl, trihydroxyphenyl, nitrohydroxyphenyl, hydroxycinnamyl and acetamidophenyl.

Suitable aldehyde indicators include, but are not limited to o-anisaldehyde, m-anisaldehyde, p-anisaldehyde, 4-acetoxybenzaldehyde, 4-acetamido benzaldehyde, 2-allyloxy benzaldehyde, 4-acetoxy-3,5-dimethoxybenz aldehyde, 4-acetoxy-3-methoxy cinnamaldehyde, α-amyl cinnamaldehyde, 9-anthraldehyde, o-aminobenzoic acid, m-aminobenzoic acid, p-aminobenzoic acid, benzaldehyde, 2-bromobenzaldehyde, 3-bromobenz aldehyde, 4-bromobenz aldehyde, 3-bromo-4-fluorobenz aldehyde, 4-bromo-2-fluorobenz aldehyde, 5-bromosalicyl aldehyde, 2-bromo-3-hydroxy-4-methoxybenz aldehyde, 5-bromo-2-hydroxy-3-methoxybenzaldehyde, 5-bromovanillin, 3-bromo-p-anisaldehyde, 5-bromo-o-anisaldehyde, 5-bromo-3-nitrosalicylaldehyde, 2,5-bis(trifluoromethyl) benzaldehyde, 3,5-bis(trifluoromethyl)benzaldehyde, α-bromocinnamaldehyde, 5-bromo-2,4-dimethoxybenzaldehyde, 5-bromoveratraldehyde, 6-bromoveratraldehyde, benzyloxy acetaldehyde, 4-butoxybenzaldehyde, 3-benzyloxybenzaldehyde, 4-benzyloxy benzaldehyde, 3-(4-methoxyphenoxy)benzaldehyde, 2-benzyloxy-3-methoxy benzaldehyde, 3-benzyloxy-4-methoxybenzaldehyde, 4-benzyloxy-3-methoxy benzaldehyde, 4-biphenylcarboxaldehyde, benzene-1,4-dicarbaldehyde, 2-benzene-1,4-dicarbaldehyde, 2-chlorobenzaldehyde, 3-chlorobenzaldehyde, 4-chlorobenzaldehyde, 2-cyanobenzaldehyde, 3-cyanobenzaldehyde, 4-cyanobenzaldehyde, 2-carboxybenzaldehyde, 3-carboxybenzaldehyde, 4-carboxybenzaldehyde, 2-chloro-4-fluorobenzaldehyde, 2-chloro-6-fluorobenzaldehyde, 3-chloro-4-fluorobenzaldehyde, 2-chloro-5-nitrobenzaldehyde, 2-chloro-6-nitrobenzaldehyde, 4-chloro-3-nitrobenzaldehyde, 5-chlorosalicylaldehyde, 5-chloro-2-nitrobenzaldehyde, cinnamaldehyde, α-chlorocinnamaldehyde, chromone-3-carboxaldehyde, cinnamaldehyde, 4-carboxybenzaldehyde, o-dimethylamino benzaldehyde, o-diethylamino benzaldehyde, o-dimethylamino cinnamaldehyde, o-diethylamino cinnamaldehyde, m-dimethylamino benzaldehyde, m-diethylamino benzaldehyde, m-dimethylamino cinnamaldehyde, m-diethylamino cinnamaldehyde, p-dimethylamino benzaldehyde, p-diethylamino benzaldehyde, p-dimethylamino cinnamaldehyde, p-diethylamino cinnamaldehyde, 2-(difluoromethoxy) benzaldehyde, 4-(difluoromethoxy) benzaldehyde, 2,3-dichlorobenzaldehyde, 2,4-dichlorobenzaldehyde, 2,6-dichlorobenzaldehyde, 3,4-dichlorobenzaldehyde, 3,5-dichlorobenzaldehyde, 2,3-difluorobenzaldehyde, 2,4-difluorobenzaldehyde, 2,5-difluorobenzaldehyde, 2,6-difluorobenzaldehyde, 3,4-difluorobenzaldehyde, 3,5-difluorobenzaldehyde, 2,3-dihydroxybenzaldehyde, 2,4-dihydroxybenzaldehyde, 2,5-dihydroxybenzaldehyde, 3,4-dihydroxybenzaldehyde, 3,5-dihydroxybenzaldehyde, 2,4-dinitrobenzaldehyde, 2,6-dinitrobenzaldehyde, 3,5-dinitrobenzaldehyde, 3,5-dibromosalicylaldehyde, 3,5-dibromo-4-hydroxy benzaldehyde, 3,5-dichloro salicylaldehyde, 3,5-diiodo salicylaldehyde, 3,4-dihydroxy-5-methoxy benzaldehyde, 2,6-dimethoxy-4-hydroxy benzaldehyde, 3,4-dimethoxy-5-hydroxy benzaldehyde, 4,6-dimethoxy salicylaldehyde, 2,3-dimethoxy benzaldehyde, 2,4-dimethoxybenzaldehyde, 2,5-dimethoxybenzaldehyde, 2,6-dimethoxybenzaldehyde, 3,5-dimethoxybenzaldehyde, 2,4-dimethylbenzaldehyde, 2,5-dimethylbenzaldehyde, 3,5-dimethylhydroxylbezaldehyde, 2,3-dimethyl-p-anisaldehyde, 2,5-dimethyl-p-anisaldehyde, 2,4-dimethoxy-3-methylbenzaldehyde, 4-(diethylamino) salicylaldehyde, diphenylacetaldehyde, 3,4-dihydroxybenzaldehyde, 3,5-dimethoxy-4-hydroxy-benzaldehyde, 2,4-dimethoxybenzaldehyde, 2,3-dimethoxybenzaldehyde, 2,5-dimethoxybenzaldehyde, 3,5-dimethoxybenzaldehyde, 3,4-dimethoxybenzaldehyde, 4-dimethylamino-1-naphthaldehyde, 4-dimethylamino-2-methoxybenzaldehyde, 2,4-dihydroxybenzaldehyde, 3,4-dihydroxybenzaldehyde, 2,5-dihydroxybenzaldehyde, 4-dibutylamino-benzaldehyde, 4-diethylamino-2-hydroxybenzaldehyde, 3,5-dimethoxy-4-hydroxy-cinnamaldehyde, 3-methoxy-4-(1-pyrrolidinyl)-benzaldehyde, 4-diethylamino-3-methoxybenzaldehyde, 3,5-dimethyl-4-hydroxybenzaldehyde, 3,5-di-tert-butyl-2-hydroxy benzaldehyde, 3,5-di-tert-butyl-4-hydroxybenzaldehyde, 3,4-dimethoxy-5-hydroxy benzaldehyde, 5-(4-(diethylamino) phenyl)-2,4-pentadienal, 3,4-dihydroxy-benzaldehyde, 3,5-dimethoxy-4-hydroxy-benzaldehyde, 3,5,-dimethyl-4-hydroxybenzaldehyde, 4-dimethylamino-2-methoxy benzaldehyde, 4-dimethylamino-1-naphthaldehyde, 2,3-dimethoxy-benzaldehyde, 2,5-dimethoxybenzaldehyde, 3,5-dimethoxy-benzaldehyde, 3,4-dimethoxy benzaldehyde, 4-dibutylamino-benzaldehyde, 4-diethylamino-2-hydroxy benzaldehyde, 3,4-dimethoxy-5-hydroxy-benzaldehyde, 5-(4-(diethylamino)phenyl)-2,4-pentadienal, 2,4-dihydroxybenzaldehyde, 4-dihydroxy-benzaldehyde, 3,4-dihydroxy-benzaldehyde, 2,5-dihydroxybenzaldehyde, 2,4-dimethoxybenzaldehyde, 3,5-dimethoxy-4-hydroxy-cinnamaldehyde, 4-diethylamino-3-methoxybenzaldehyde, 2-ethoxybenzaldehyde, 4-ethoxybenzaldehyde, 3-ethoxy-4-hydroxybenzaldehyde, 3-ethoxysalicylaldehyde, 3-ethoxy-4-hydroxy benzaldehyde, N-ethylcarbazole-3-aldehyde, 3-ethoxy-4-methoxy benzaldehyde, 4-ethoxy benzaldehyde, 4-ethoxy benzaldehyde, 3-ethoxy-4-hydroxy benzaldehyde, 2-fluoro benzaldehyde, 3-fluorobenzaldehyde, 4-fluorobenzaldehyde, 2-formylphenylboronic acid, 3-formylphenylboronic acid, 4-formylphenylboronic acid, 2-formylbenzenesulfonic acid, 2-fluoro-5-nitrobenzaldehyde, 3-fluorosalicylaldehyde, 4-formyl-1,3-benzenedisulfonic acid, 2-fluoro-3-(trifluoromethyl)benzaldehyde, 2-fluoro-6-(trifluoromethyl)benzaldehyde, 4-fluoro-2-(trifluoromethyl) benzaldehyde, 4-fluoro-3-(trifluoromethyl)benzaldehyde, 3-fluoro-p-anisaldehyde, 3-fluoro-2-methlbenzaldehyde, 4-ethylbenzaldehyde, 2-fluorenecarboxaldehyde, 3-hydroxybenzaldehyde, 4-hydroxy benzaldehyde, 2-hydroxy-5-nitrobenzaldehyde, 3-hydroxy-4-nitro benzaldehyde, 4-hydroxy-3-nitro benzaldehyde, 5-hydroxy-2-nitrobenzaldehyde, 2-hydroxy-4-methoxybenzaldehyde, 2-hydroxy-5-methoxybenzaldehyde, 3-hydroxy-4-methoxybenzaldehyde, 2-hydroxy-5-methyl-1,3-benzenedicarboxaldehyde, hydrocinnamaldehyde, 2-hydroxy-1-naphthaldehyde, 4-(hexyloxy)benzaldehyde, 4-hydroxy-3-methoxybenzaldehyde (vanillin), 3-hydroxy-4-methoxy-benzaldehyde (isovanillin), 4-hydroxybenzaldehyde, 4-hydroxy-2-methoxybenzaldehyde, 2-hydroxybenzaldehyde, 4-hydroxy-1-naphthaldehyde, 4-methoxy-1-naphthaldehyde, 4′-hydroxy-biphenyl-1-carbaldehyde, 2-hydroxy-3-methoxybenzaldehyde, 4-hydroxy-3-methoxy-cinnamaldehyde, 6-hydroxychromen-3-carbox-aldehyde, α-hexylcinnamaldehyde, 4-hydroxy-3-methoxy-cinnamaldehyde, 4-hydroxy-benzaldehyde, 4-hydroxy-2-methoxy-benzaldehyde, 2-hydroxybenzaldehyde, 4-hydroxy-1-naphthaldehyde, 4′-hydroxy-biphenyl-1-carbaldehyde, 2-hydroxy-3-methoxybenzaldehyde, isophthalaldehyde, 5-iodovanillin, 4-isopropylbenzaldehyde, indole-3-carbaldehyde, 6-methyl-2-pyridinecarboxaldehyde, 2,3-(methylenedioxy) benzaldehyde, 3-methoxy-5-nitrosalicylaldehyde, 3-methyl-p-anisaldehyde, 2-methoxycinnamaldehyde, mesitaldehyde, 2-methoxy-1-naphthaldehyde, N-methylpyrrole-2-aldehyde, 5-methylfurfural, 6-methylindole-3-carboxaldehyde, 6-methyl-4-oxo-1 (4H)-benzopyran-3-carbaldehyde, 2-methyl-1,4-naphthoquinone, 4-carboxybenzaldehyde, 4-methoxy-1-naphthalaldehyde, methyl 2-formyl-3,5-dimethoxybenzoate, 2-methoxy-1-naphthalaldehyde, 4-methyl-5-imidazole-carboxaldehyde, 2-methyl-1,4-naphthoquinone, 3-methoxy-4-(1-pyrrolidinyl)-benzaldehyde, 2-methoxy-1-naphthaldehyde, 4-methoxy-1-naphthaldehyde, 2-nitrobenzaldehyde, 3-nitrobenzaldehyde, 4-nitrobenzaldehyde, 5-nitro-vanillin, 2-nitrocinnamaldehyde, 4-nitrocinnamaldehyde, 2-nitrobenzaldehyde, 3-nitrobenzaldehyde, 4-nitrobenzaldehyde. 6-nitroveratraldehyde, 1-naphthaldehyde, 2-naphthaldehyde, 2,3-naphthalendicarboxaldehyde, 1,8-naphthalaldehydic acid, 2-nitrobenzaldehyde, 3-nitrobenzaldehyde, 4-nitrobenzaldehyde, phenylacetaldehyde, 2-pyridinecarboxaldehyde, 3-pyridinecarboxaldehyde, 4-pyridinecarboxaldehyde, 4-pyridinecarboxaldehyde N-oxide, phthalaldehyde, piperonal, 2-phenyl propionaldehyde, pentafluorobenzaldehyde, 3-phenylbutyraldehyde, 4-propoxybenzaldehyde, pentamethylbenzaldehyde, 3-phenoxybenzaldehyde, 4-phenoxybenzaldehyde, phenylpropargyl aldehyde, 1,2-phthaldialdehyde, pyrrole-2-aldehyde, phthalimidoacetaldehyde, o-phtalaldehyde, 1,2-phthaldialdehyde, 2-quinolinecarboxaldehyde, 3-quinolinecarboxaldehyde, 4-quinolinecarboxaldehyde, salicylaldehyde, syringaldeyde, o-tolualdehyde, m-tolualdehyde, p-tolualdehyde, 3-(trifluoromethoxy)benzaldehyde, 4-(trifluoromethoxy)benzaldehyde α,α,α-trifluoro-o-tolualdehyde α,α,α-trifluoro-m-tolualdehyde, α,α,α-trifluoro-p-tolualdehyde, terephthalaldehyde, 5-(trifluoromethoxy)salicylaldehyde, 2,3,5-trichloro benzaldehyde, 2,3,6-trichlorobenzaldehyde, 2,3,4-trifluorobenzaldehyde, 2,3,6-trifluoro benzaldehyde, 2,3,4-trihydroxy benzaldehyde, 2,4,6-trihydroxybenzaldehyde, 3,4,5-trihydroxy benzaldehyde, 2,3,5,6-tetrafluorobenzaldehyde, 2,3,4-trimethoxy benzaldehyde, 2,4,5-trimethoxybenzaldehyde, 2,4,6-trimethoxybenzaldehyde, 3,4,5-trimethoxy benzaldehyde, 4-4-tert-butylbenzaldehyde, 3-tert-butyl-2-hydroxy benzaldehyde, 5-tert-butyl-2-hydroxy benzaldehyde, 2,3,4-trihydroxy benzaldehyde, 3,4,5-trihydroxybenzaldehyde, 2,3-thiophene-dicarboxaldehyde, 2,5-thiophene-dicarboxaldehyde, thiophene-2-aldehyde, thiophene-3-aldehyde, 2,4,6-trihydroxy benzaldehyde, 2,3,4-trihydroxybenzaldehyde, 3,4,5-trihydroxybenzaldehyde, 2,4,6-trihydroxy benzaldehyde, vanillin, o-vanillin, vinyl benzaldehyde, veratraldehyde and vanillin acetate.

According to a feature of the present invention, the indicator is an aromatic aldehyde of the structure Ar—(CR_{n,a}═CR_{n,b})ₙ—CH═O where: n is an integer 0 or greater; Ar is aromatic; and each one of R_{n,a} and R_{n,b} are independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl, cyano, nitro, amino and —NR₁₅R₁₆, where R₁₅ and R₁₆ are independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl and cyano, or, alternatively, at least two of substituents of Ar, R₁₅, R₁₆, R_{n,a} and/or R_{n,b} form an aromatic, heteroaromatic, alicyclic or heteroalicyclic ring. According to a feature of the present invention, the aromatic group Ar is substituted with at least one positive charge stabilizing functional group, preferably para to the —(CR_{n,a}═CR_{n,b})ₙ—CH═O group. Preferred positive charge stabilizing functional groups include but are not limited to amines, alkylamines, dialkylamines, alcohols, esters, amides, acids and alkyls. In the currently known best mode of the present invention, at least one of the at least one positive charge stabilizing functional groups is a disubstituted amine, preferably a dialkyl amine, preferably para to the —(CR_{n,a}═CR_{n,b})ₙ—CH═O group. Suitable aromatic aldehydes include but are not limited to p-DMAC, p-DMAB, p-DEAC and p-DEAB.

Generally, the preferred concentration of an indicator in an indicator solution is not critical to the practice of the teachings of the present invention. That said, according to a feature of the present invention, the concentration of the indicator in the indicator solution is less than about 4% by weight, less than about 1% by weight and even less than about 0.5% by weight. According to a feature of the present invention, the concentration of the indicator in the indicator solution is greater than about 0.1% by weight.

According to an embodiment of the method of the present invention, the sample is collected and/or concentrated for testing.

In one embodiment, collecting or concentrating is performed prior to contacting with the indicator solution. In one embodiment of the present invention, by collecting and/or concentrating is meant gathering the sample from an area, for example by wiping an area where the sample is found with a collector and/or a concentrator. Suitable collectors and/or concentrators include but are not limited to bibulous materials, cloth, fabrics, felt, flannel, membranes, pads, papers, sponges, swabs, swatches, tissues and wipes.

According to a feature of the present invention, the contacting of the sample with the indicator solution occurs on the collector and/or the concentrator. In one embodiment, the collector and/or the concentrator is impregnated with the indicator solution prior to the wiping, e.g. a “wet-wipe”. In another embodiment, the indicator is applied onto the collector and/or the concentrator prior to the wiping. In a further embodiment, the indicator is applied onto the collector and/or the concentrator subsequent to the wiping.

According to a feature of the present invention, the contacting of the sample with the indicator solution occurs on a location where the sample is found, by applying the indicator solution to the location.

According to a feature of the present invention, applying an indicator solution includes dispensing the indicator from a vessel. Suitable vessels include but are not limited to aerosol dispensers, bags, beakers, bottles, droppers, jars, mechanically actuated spray dispensers, pressurized spray dispensers, pump spray dispensers, sacks, sachets, spray dispensers, squeeze bottle, ampoules, syringes, capsules, foil wrappers and tubes.

According to a feature of the present invention there is provided a kit for determining the presence or absence of an uronium salt (e.g. urea nitrate) in a sample comprising: a) at least one indicator selected from the group consisting of an aldehyde and a compound configured to form an aromatic dialkylaminium ion upon reaction with the uronium salt; b) a device selected from amongst a collector, a concentrator or a combination thereof; and c) a vessel configured to hold and dispense a solution of the indicator having a pH of greater than about 2. According to a feature of a kit of the present invention, the indicator is packaged in a packaging material and identified in print, in or on the packaging material, for use for determining the presence or absence of an uronium salt in a sample.

According to a feature of the present invention there is also provided a kit for determining the presence or absence of an explosive in a sample (e.g. wherein the explosive comprises an uronium salt such as urea nitrate): a) at least one indicator selected from the group consisting of an aldehyde and a compound configured to form an aromatic dialkylaminium ion upon reaction with the uronium salt; b) a device selected from amongst a collector, a concentrator or a combination thereof; and c) a vessel configured to hold and dispense a solution of the indicator having a pH of greater than about 2. According to a feature of a kit of the present invention, the indicator is packaged in a packaging material and identified in print, in or on the packaging material, for use for determining the presence or absence of an explosive in a sample.

According to a feature of a kit of the present invention, the indicator is provided in a concentrated form or as a ready-to-use indicator solution.

Preferred concentrated forms include a substantially pure indicator in solid or liquid form, or a solid or liquid containing a high percentage of the indicator, e.g. greater than about 1% by weight, greater than about 5% by weight, greater than about 10% by weight, greater than about 20% by weight, greater than about 30% by weight, greater than about 50% by weight or even greater than about 80% by weight of indicator.

When the indicator is provided in concentrated form in a kit of the present invention, it is preferable to also provide a solvent as part of the kit. According to a feature of the present invention, the type and amount of solvent provided is chosen so that upon mixing with the concentrated form of the indicator, an indicator solution having a pH of greater than about 2 is made. According to a feature of the present invention, an indicator solution having a pH greater than about 3, greater than about 4, greater than about 5, greater than about 6 and even greater than about 7 is made.

When the indicator is provided in a kit of the present invention as a ready-to-use indicator solution, the indicator solution includes at the least indicator and a solvent, and is of a pH greater than about 2. According to a feature of the present invention, an indicator solution has a pH greater than about 3, greater than about 4, greater than about 5, greater than about 6 and even greater than about 7. Generally, the preferred concentration of an indicator in a ready-to-use indicator solution in a kit of the present invention is not critical. That said, according to a feature of the present invention, the concentration of the indicator in the indicator solution is less than about 4% by weight, less than about 1% by weight and even less than about 0.5% by weight. According to a feature of the present invention, the concentration of the indicator in the indicator solution is greater than about 0.1% by weight.

According to a feature of the present invention, the solvent in a ready-to-use indicator solution in a kit of the present invention or provided separately from the indicator as part of a kit of the present invention includes at least one protic solvent. Suitable protic solvents include but are not limited to water, alcohol, ethanol, methanol, propanol, isopropanol, butanol, isoamylalcohol, glycol and 1,2-dihydroxypropane.

According to a feature of the present invention, the solvent in a ready-to-use indicator solution in a kit of the present invention or provided separately from the indicator as part of a kit of the present invention includes, in addition to a first protic solvent, a second solvent, the second solvent being more volatile than the first protic solvent. Preferably the volatility of the second solvent is such that it evaporates when the indicator solution and the sample are contacted, so as to increase the concentration of the sample in the indicator solution. Suitable second solvents include volatile solvents including but not limited to ethanol, methanol, butyl acetate, acetonitrile, chloroform, dichloromethane, diethyl ether and ethyl acetate. In such a case, a preferred first protic solvent is water.

According to a feature of the present invention, the solvent in a ready-to-use indicator solution in a kit of the present invention or provided separately from the indicator as part of a kit of the present invention includes, in addition to at least one protic solvent, at least one aprotic solvent. Suitable aprotic solvents include but are not limited to ethers, acetates, ketones, acetone, butyl acetate, acetonitrile, chloroform, dichloromethane, diethyl ether, dimethyl formamide, dimethylsulfoxide, ethyl acetate and tetrahydrofuran.

According to a feature of the present invention, the solvent in a ready-to-use indicator solution in a kit of the present invention or provided separately from the indicator as part of a kit of the present invention is a mixture of ethanol and water.

According to a feature of the present invention, a collector and/or a concentrator provided with a kit of the present invention is selected from the group consisting of bibulous materials, cloth, fabrics, felt, flannel, membranes, pads, papers, sponges, swabs, swatches, tissues and wipes.

According to a feature of the present invention, a collector and/or a concentrator provided with a kit of the present invention is impregnated with the indicator.

According to feature of the present invention, the vessel provided with a kit of the present invention is selected from the group including but not limited to aerosol dispensers, bags, beakers, bottles, droppers, jars, mechanically actuated spray dispensers, pressurized spray dispensers, pump spray dispensers, sacks, sachet, spray dispensers, squeeze bottle, ampoules, syringes, capsules, foil wrappers and tubes.

According to a feature of the present invention, an indicator provided with a kit of the present invention is selected from the group including but not limited to p-dialkylaminocinnamaldehydes, p-dialkylaminocinnamyl ketones, linear p-dialkylaminocinnamic acids, cyclic p-dialkylaminocinnamic acids (coumarins), linear p-dialkylaminocinnamamides, cyclic p-dialkylaminocinnamamides (quinolinones), p-dialkylaminobenzaldehydes, p-dialkylaminophenyl ketones, p-dialkylaminobenzoic acids, p-dialkylamino benzamides, salts thereof and esters thereof.

According to a feature of the present invention, an indicator provided with a kit of the present invention is an aldehyde of the structure R—CHO wherein R is selected from the group consisting of alkyl, hydroxyalkyl, trihaloalkyl, cycloalkyl, alkenyl, alkynyl, aryl, benzyl, heteroaryl, heteroalicyclic, aminobenzyl, alkylaminobenzyl, dialkylaminobenzyl, dialkoxyaminobenzyl, cinnamyl, aminocinnamyl, alkyaminocinnamyl, dialkyaminocinnamyl, alkoxyaminocinnamyl, dialkoxyaminocinnamyl, nitrobenzyl, alkylbenzyl, alkoxybenzyl, phenol, dihydroxyphenyl, trihydroxyphenyl, nitrohydroxyphenyl, hydroxycinnamyl and acetamidophenyl.

According to a feature of the present invention, an indicator provided with a kit of the present invention is an aldehyde selected from the group including but not limited to o-anisaldehyde, m-anisaldehyde, p-anisaldehyde, 4-acetoxybenzaldehyde, 4-acetamido benzaldehyde, 2-allyloxy benzaldehyde, 4-acetoxy-3,5-dimethoxybenz aldehyde, 4-acetoxy-3-methoxy cinnamaldehyde, α-amyl cinnamaldehyde, 9-anthraldehyde, o-aminobenzoic acid, m-aminobenzoic acid, p-aminobenzoic acid, benzaldehyde, 2-bromobenzaldehyde, 3-bromobenz aldehyde, 4-bromobenz aldehyde, 3-bromo-4-fluorobenz aldehyde, 4-bromo-2-fluorobenz aldehyde, 5-bromosalicyl aldehyde, 2-bromo-3-hydroxy-4-methoxybenz aldehyde, 5-bromo-2-hydroxy-3-methoxybenzaldehyde, 5-bromovanillin, 3-bromo-p-anisaldehyde, 5-bromo-o-anisaldehyde, 5-bromo-3-nitrosalicylaldehyde, 2,5-bis(trifluoromethyl) benzaldehyde, 3,5-bis(trifluoromethyl)benzaldehyde, α-bromocinnamaldehyde, 5-bromo-2,4-dimethoxybenzaldehyde, 5-bromoveratraldehyde, 6-bromoveratraldehyde, benzyloxy acetaldehyde, 4-butoxybenzaldehyde, 3-benzyloxybenzaldehyde, 4-benzyloxy benzaldehyde, 3-(4-methoxyphenoxy)benzaldehyde, 2-benzyloxy-3-methoxy benzaldehyde, 3-benzyloxy-4-methoxybenzaldehyde, 4-benzyloxy-3-methoxy benzaldehyde, 4-biphenylcarboxaldehyde, benzene-1,4-dicarbaldehyde, 2-benzene-1,4-dicarbaldehyde, 2-chlorobenzaldehyde, 3-chlorobenzaldehyde, 4-chlorobenzaldehyde, 2-cyanobenzaldehyde, 3-cyanobenzaldehyde, 4-cyanobenzaldehyde, 2-carboxybenzaldehyde, 3-carboxybenzaldehyde, 4-carboxybenzaldehyde, 2-chloro-4-fluorobenzaldehyde, 2-chloro-6-fluorobenzaldehyde, 3-chloro-4-fluorobenzaldehyde, 2-chloro-5-nitrobenzaldehyde, 2-chloro-6-nitrobenzaldehyde, 4-chloro-3-nitrobenzaldehyde, 5-chlorosalicylaldehyde, 5-chloro-2-nitrobenzaldehyde, cinnamaldehyde, α-chlorocinnamaldehyde, chromone-3-carboxaldehyde, cinnamaldehyde, 4-carboxybenzaldehyde, o-dimethylamino benzaldehyde, o-diethylamino benzaldehyde, o-dimethylamino cinnamaldehyde, o-diethylamino cinnamaldehyde, m-dimethylamino benzaldehyde, m-diethylamino benzaldehyde, m-dimethylamino cinnamaldehyde, m-diethylamino cinnamaldehyde, p-dimethylamino benzaldehyde, p-diethylamino benzaldehyde, p-dimethylamino cinnamaldehyde, p-diethylamino cinnamaldehyde, 2-(difluoromethoxy) benzaldehyde, 4-(difluoromethoxy) benzaldehyde, 2,3-dichlorobenzaldehyde, 2,4-dichlorobenzaldehyde, 2,6-dichlorobenzaldehyde, 3,4-dichlorobenzaldehyde, 3,5-dichlorobenzaldehyde, 2,3-difluorobenzaldehyde, 2,4-difluorobenzaldehyde, 2,5-difluorobenzaldehyde, 2,6-difluorobenzaldehyde, 3,4-difluorobenzaldehyde, 3,5-difluorobenzaldehyde, 2,3-dihydroxybenzaldehyde, 2,4-dihydroxybenzaldehyde, 2,5-dihydroxybenzaldehyde, 3,4-dihydroxybenzaldehyde, 3,5-dihydroxybenzaldehyde, 2,4-dinitrobenzaldehyde, 2,6-dinitrobenzaldehyde, 3,5-dinitrobenzaldehyde, 3,5-dibromosalicylaldehyde, 3,5-dibromo-4-hydroxy benzaldehyde, 3,5-dichloro salicylaldehyde, 3,5-diiodo salicylaldehyde, 3,4-dihydroxy-5-methoxy benzaldehyde, 2,6-dimethoxy-4-hydroxy benzaldehyde, 3,4-dimethoxy-5-hydroxy benzaldehyde, 4,6-dimethoxy salicylaldehyde, 2,3-dimethoxy benzaldehyde, 2,4-dimethoxybenzaldehyde, 2,5-dimethoxybenzaldehyde, 2,6-dimethoxybenzaldehyde, 3,5-dimethoxybenzaldehyde, 2,4-dimethylbenzaldehyde, 2,5-dimethylbenzaldehyde, 3,5-dimethylhydroxylbezaldehyde, 2,3-dimethyl-p-anisaldehyde, 2,5-dimethyl-p-anisaldehyde, 2,4-dimethoxy-3-methylbenzaldehyde, 4-(diethylamino) salicylaldehyde, diphenylacetaldehyde, 3,4-dihydroxybenzaldehyde, 3,5-dimethoxy-4-hydroxy-benzaldehyde, 2,4-dimethoxybenzaldehyde, 2,3-dimethoxybenzaldehyde, 2,5-dimethoxybenzaldehyde, 3,5-dimethoxybenzaldehyde, 3,4-dimethoxybenzaldehyde, 4-dimethylamino-1-naphthaldehyde, 4-dimethylamino-2-methoxybenzaldehyde, 2,4-dihydroxybenzaldehyde, 3,4-dihydroxybenzaldehyde, 2,5-dihydroxybenzaldehyde, 4-dibutylamino-benzaldehyde, 4-diethylamino-2-hydroxybenzaldehyde, 3,5-dimethoxy-4-hydroxy-cinnamaldehyde, 3-methoxy-4-(1-pyrrolidinyl)-benzaldehyde, 4-diethylamino-3-methoxybenzaldehyde, 3,5-dimethyl-4-hydroxybenzaldehyde, 3,5-di-tert-butyl-2-hydroxy benzaldehyde, 3,5-di-tert-butyl-4-hydroxybenzaldehyde, 3,4-dimethoxy-5-hydroxy benzaldehyde, 5-(4-(diethylamino) phenyl)-2,4-pentadienal, 3,4-dihydroxy-benzaldehyde, 3,5-dimethoxy-4-hydroxy-benzaldehyde, 3,5,-dimethyl-4-hydroxybenzaldehyde, 4-dimethylamino-2-methoxy benzaldehyde, 4-dimethylamino-1-naphthaldehyde, 2,3-dimethoxy-benzaldehyde, 2,5-dimethoxybenzaldehyde, 3,5-dimethoxy-benzaldehyde, 3,4-dimethoxy benzaldehyde, 4-dibutylamino-benzaldehyde, 4-diethylamino-2-hydroxy benzaldehyde, 3,4-dimethoxy-5-hydroxy-benzaldehyde, 5-(4-(diethylamino)phenyl)-2,4-pentadienal, 2,4-dihydroxybenzaldehyde, 4-dihydroxy-benzaldehyde, 3,4-dihydroxy-benzaldehyde, 2,5-dihydroxybenzaldehyde, 2,4-dimethoxybenzaldehyde, 3,5-dimethoxy-4-hydroxy-cinnamaldehyde, 4-diethylamino-3-methoxybenzaldehyde, 2-ethoxybenzaldehyde, 4-ethoxybenzaldehyde, 3-ethoxy-4-hydroxybenzaldehyde, 3-ethoxysalicylaldehyde, 3-ethoxy-4-hydroxy benzaldehyde, N-ethylcarbazole-3-aldehyde, 3-ethoxy-4-methoxy benzaldehyde, 4-ethoxy benzaldehyde, 4-ethoxy benzaldehyde, 3-ethoxy-4-hydroxy benzaldehyde, 2-fluoro benzaldehyde, 3-fluorobenzaldehyde, 4-fluorobenzaldehyde, 2-formylphenylboronic acid, 3-formylphenylboronic acid, 4-formylphenylboronic acid, 2-formylbenzenesulfonic acid, 2-fluoro-5-nitrobenzaldehyde, 3-fluorosalicylaldehyde, 4-formyl-1,3-benzenedisulfonic acid, 2-fluoro-3-(trifluoromethyl)benzaldehyde, 2-fluoro-6-(trifluoromethyl)benzaldehyde, 4-fluoro-2-(trifluoromethyl) benzaldehyde, 4-fluoro-3-(trifluoromethyl)benzaldehyde, 3-fluoro-p-anisaldehyde, 3-fluoro-2-methlbenzaldehyde, 4-ethylbenzaldehyde, 2-fluorenecarboxaldehyde, 3-hydroxybenzaldehyde, 4-hydroxy benzaldehyde, 2-hydroxy-5-nitrobenzaldehyde, 3-hydroxy-4-nitro benzaldehyde, 4-hydroxy-3-nitro benzaldehyde, 5-hydroxy-2-nitrobenzaldehyde, 2-hydroxy-4-methoxybenzaldehyde, 2-hydroxy-5-methoxybenzaldehyde, 3-hydroxy-4-methoxybenzaldehyde, 2-hydroxy-5-methyl-1,3-benzenedicarboxaldehyde, hydrocinnamaldehyde, 2-hydroxy-1-naphthaldehyde, 4-(hexyloxy)benzaldehyde, 4-hydroxy-3-methoxybenzaldehyde (vanillin), 3-hydroxy-4-methoxy-benzaldehyde (isovanillin), 4-hydroxybenzaldehyde, 4-hydroxy-2-methoxybenzaldehyde, 2-hydroxybenzaldehyde, 4-hydroxy-1-naphthaldehyde, 4-methoxy-1-naphthaldehyde, 4′-hydroxy-biphenyl-1-carbaldehyde, 2-hydroxy-3-methoxybenzaldehyde, 4-hydroxy-3-methoxy-cinnamaldehyde, 6-hydroxychromen-3-carbox-aldehyde, α-hexylcinnamaldehyde, 4-hydroxy-3-methoxy-cinnamaldehyde, 4-hydroxy-benzaldehyde, 4-hydroxy-2-methoxy-benzaldehyde, 2-hydroxybenzaldehyde, 4-hydroxy-1-naphthaldehyde, 4′-hydroxy-biphenyl-1-carbaldehyde, 2-hydroxy-3-methoxybenzaldehyde, isophthalaldehyde, 5-iodovanillin, 4-isopropylbenzaldehyde, indole-3-carbaldehyde, 6-methyl-2-pyridinecarboxaldehyde, 2,3-(methylenedioxy) benzaldehyde, 3-methoxy-5-nitrosalicylaldehyde, 3-methyl-p-anisaldehyde, 2-methoxycinnamaldehyde, mesitaldehyde, 2-methoxy-1-naphthaldehyde, N-methylpyrrole-2-aldehyde, 5-methylfurfural, 6-methylindole-3-carboxaldehyde, 6-methyl-4-oxo-1 (4H)-benzopyran-3-carbaldehyde, 2-methyl-1,4-naphthoquinone, 4-carboxybenzaldehyde, 4-methoxy-1-naphthalaldehyde, methyl 2-formyl-3,5-dimethoxybenzoate, 2-methoxy-1-naphthalaldehyde, 4-methyl-5-imidazole-carboxaldehyde, 2-methyl-1,4-naphthoquinone, 3-methoxy-4-(1-pyrrolidinyl)-benzaldehyde, 2-methoxy-1-naphthaldehyde, 4-methoxy-1-naphthaldehyde, 2-nitrobenzaldehyde, 3-nitrobenzaldehyde, 4-nitrobenzaldehyde, 5-nitro-vanillin, 2-nitrocinnamaldehyde, 4-nitrocinnamaldehyde, 2-nitrobenzaldehyde, 3-nitrobenzaldehyde, 4-nitrobenzaldehyde. 6-nitroveratraldehyde, 1-naphthaldehyde, 2-naphthaldehyde, 2,3-naphthalendicarboxaldehyde, 1,8-naphthalaldehydic acid, 2-nitrobenzaldehyde, 3-nitrobenzaldehyde, 4-nitrobenzaldehyde, phenylacetaldehyde, 2-pyridinecarboxaldehyde, 3-pyridinecarboxaldehyde, 4-pyridinecarboxaldehyde, 4-pyridinecarboxaldehyde N-oxide, phthalaldehyde, piperonal, 2-phenyl propionaldehyde, pentafluorobenzaldehyde, 3-phenylbutyraldehyde, 4-propoxybenzaldehyde, pentamethylbenzaldehyde, 3-phenoxybenzaldehyde, 4-phenoxybenzaldehyde, phenylpropargyl aldehyde, 1,2-phthaldialdehyde, pyrrole-2-aldehyde, phthalimidoacetaldehyde, o-phtalaldehyde, 1,2-phthaldialdehyde, 2-quinolinecarboxaldehyde, 3-quinolinecarboxaldehyde, 4-quinolinecarboxaldehyde, salicylaldehyde, syringaldeyde, o-tolualdehyde, m-tolualdehyde, p-tolualdehyde, 3-(trifluoromethoxy)benzaldehyde, 4-(trifluoromethoxy)benzaldehyde α,α,α-trifluoro-o-tolualdehyde α,α,α-trifluoro-m-tolualdehyde, α,α,α-trifluoro-p-tolualdehyde, terephthalaldehyde, 5-(trifluoromethoxy)salicylaldehyde, 2,3,5-trichloro benzaldehyde, 2,3,6-trichlorobenzaldehyde, 2,3,4-trifluorobenzaldehyde, 2,3,6-trifluoro benzaldehyde, 2,3,4-trihydroxy benzaldehyde, 2,4,6-trihydroxybenzaldehyde, 3,4,5-trihydroxy benzaldehyde, 2,3,5,6-tetrafluorobenzaldehyde, 2,3,4-trimethoxy benzaldehyde, 2,4,5-trimethoxybenzaldehyde, 2,4,6-trimethoxybenzaldehyde, 3,4,5-trimethoxy benzaldehyde, 4-4-tert-butylbenzaldehyde, 3-tert-butyl-2-hydroxy benzaldehyde, 5-tert-butyl-2-hydroxy benzaldehyde, 2,3,4-trihydroxy benzaldehyde, 3,4,5-trihydroxybenzaldehyde, 2,3-thiophene-dicarboxaldehyde, 2,5-thiophene-dicarboxaldehyde, thiophene-2-aldehyde, thiophene-3-aldehyde, 2,4,6-trihydroxy benzaldehyde, 2,3,4-trihydroxybenzaldehyde, 3,4,5-trihydroxybenzaldehyde, 2,4,6-trihydroxy benzaldehyde, vanillin, o-vanillin, vinyl benzaldehyde, veratraldehyde and vanillin acetate.

According to a feature of the present invention, an indicator provided with a kit of the present invention is an aromatic aldehyde of the structure Ar—(CR_{n,a}═CR_{n,b})ₙ—CH═O where: n is an integer 0 or greater; Ar is aromatic; and each one of R_{n,a} and R_{n,b} are independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl, cyano, nitro, amino and —NR₁₅R₁₆, where R₁₅ and R₁₆ are independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl and cyano, or, alternatively, at least two of substituents of Ar, R₁₅, R₁₆, R_{n,a} and/or R_{n,b} form an aromatic, heteroaromatic, alicyclic or heteroalicyclic ring. According to a feature of the present invention, the aromatic group Ar is substituted with at least one positive charge stabilizing functional group, preferably para to the —(CR_{n,a}═CR_{n,b})ₙ—CH═O group. Preferred positive charge stabilizing functional groups include but are not limited to amines, alkylamines, dialkylamines, alcohols, esters, amides, acids and alkyls. In the currently known best mode of the present invention, at least one of the at least one positive charge stabilizing functional groups is a disubstituted amine, preferably a dialkyl amine, preferably para to the —(CR_{n,a}═CR_{n,b})ₙ—CH═O group. Suitable aromatic aldehydes include but are not limited to p-DMAC, p-DMAB, p-DEAC and p-DEAB.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### DESCRIPTION OF THE PREFERRED EMBODIMENTS

The present invention provides a colorimetric method useful in the quick and efficient identification of uronium salts, e.g., urea nitrate. The present invention also provides a kit useful in implementing the method of the present invention.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The principles and uses of the present invention may be better understood with reference to the Examples and accompanying descriptions. Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The present invention is a colorimetric method, based on the use of an indicator (such as p-DMAC, p-DEAC, p-DMAB, p-DEAB and mixtures thereof) that reacts efficiently with an amine (such as urea) in very acidic solution. However, unlike in prior art methods, the indicator is provided in a solution that is not very acidic.

According to the present invention, the presence or absence of an uronium salt in a sample is preferably determined by the occurrence of a chromogenic reaction of the uronium salt with at least one indicator in an indicator solution having a pH greater than about 2, where the indicator is either or both an aldehyde and a compound configured to form an aromatic disubstituted aminium ion upon reaction with an uronium salt.

When a sample containing an uronium salt is contacted with an indicator solution in accordance with the teachings of the present invention, the cationizing moiety associated with the ureic moiety acidifies the indicator solution to the point that the desired chromogenic reaction occurs quickly, e.g. preferably within a few seconds to a few minutes.

When a sample containing common interferants such as LSD or urea is contacted with an indicator solution according to the method of the present invention, the pH of the indicator solution does not change or even becomes somewhat less acidic. A chromogenic reaction occurs very slowly, if at all.

Generally the lower limit of detection and robustness of the method of the present invention is determined by the pH of the indicator solution and the reactivity of the indicator itself.

Indicators useful in implementing the present invention generally react with certain amines in solutions having a pH of less than 2. Thus, an indicator solution used in implementing the present invention has a pH of greater than about 2. It is preferred, however, to use an indicator solution having a higher pH. Lower pH indicator solutions can be irritants and unhealthy. Further, the robustness against false positives is somewhat increased when using indicator solutions having a higher pH. Lastly, it is generally simplest to combine an indicator with a solvent to make an indicator solution without the added expense and effort of modifying the pH. It is therefore generally preferred that the pH of an indicator solution be the unadjusted pH of the indicator in a solvent. Thus, although an indicator solution of the present invention has a pH greater than about 2, preferably the pH is greater than about 3, more preferably greater than about 4, more preferably greater than about 5, more preferably greater than about 6 and even more preferably greater than about 7.

As stated above, an indicator solution useful in implementing the present invention includes at least one indicator that is either or both an aldehyde and a compound configured to form an aromatic disubstituted aminium ion upon reaction with the urea.

Certain compounds used in implementing the method of the present invention can exist as salts. Certain compounds used in implementing the method of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds used in implementing the method of the present invention possess asymmetric carbon atoms (optical centers) or double bonds; the racemates, diastereomers, geometric isomers and individual isomers are encompassed within the scope of the present invention.

Compounds used in implementing the method of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein in the specification and in the claims section that follows, the term “alkyl” refers to a saturated aliphatic hydrocarbon including straight chain and branched chain groups. Preferably, the alkyl group has 1 to 20 carbon atoms. Whenever a numerical range; e.g., “1-20”, is stated herein, it means that the group, in this case the alkyl group, may contain 1 carbon atom, 2 carbon atoms, 3 carbon atoms, etc., up to and including 20 carbon atoms. More preferably, it is a medium size alkyl having 1 to 10 carbon atoms. Most preferably, it is a lower alkyl having 1 to 4 carbon atoms. The alkyl group may be substituted or unsubstituted. When substituted, the substituent group can be, for example, cycloalkyl, aryl, heteroaryl, heteroalicyclic, hydroxy, alkoxy, aryloxy, thiohydroxy, thioalkoxy, thioaryloxy, cyano, halo, carbonyl, thiocarbonyl, O-carbamyl, N-carbamyl, O-thiocarbamyl, N-thiocarbamyl, C-amido, N-amido, C-carboxy, O-carboxy, nitro, sulfonamido, trihalomethanesulfonamido, silyl, guanyl, guanidino, ureido, amino or NR^{a}R^{b}, wherein Rₐ and R_{b} are each independently hydrogen, alkyl, cycloalkyl, aryl, carbonyl, sulfonyl, trihalomethysulfonyl and, combined, a five- or six-member heteroalicyclic ring.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, aryl, heteroaryl, heteroalicyclic, hydroxy, alkoxy, aryloxy, thiohydroxy, thioalkoxy, thioaryloxy, cyano, halo, carbonyl, thiocarbonyl, C-carboxy, O-carboxy, O-carbamyl, N-carbamyl, C-amido, N-amido, nitro, amino and NRₐR_{b}.

An “alkenyl” group refers to an alkyl group which consists of at least two carbon atoms and at least one carbon-carbon double bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, halo, trihalomethyl, alkyl, hydroxy, alkoxy, aryloxy, thiohydroxy, thiocarbonyl, C-carboxy, O-carboxy, O-carbamyl, N-carbamyl, O-thiocarbamyl, N-thiocarbamyl, C-amido, N-amido, sulfinyl, sulfonyl, amino and NRₐR_{b} as defined above.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, cycloalkyl, halo, trihalomethyl, hydroxy, alkoxy, aryloxy, thiohydroxy, thiocarbonyl, sulfonamido, C-carboxy, O-carboxy, sulfinyl, sulfonyl, O-carbamyl, N-carbamyl, O-thiocarbamyl, N-thiocarbamyl, C-amido, N-amido, amino or NRₐR_{b} as defined above.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, alkyl, cycloalkyl, aryl, heteroaryl, halo, trihalomethyl, hydroxy, alkoxy, aryloxy, thiohydroxy, thioalkoxy, thioaryloxy, cyano, nitro, carbonyl, thiocarbonyl, C-carboxy, O-carboxy, O-carbamyl, N-carbamyl, O-thiocarbamyl, N-thiocarbamyl, sulfinyl, sulfonyl, C-amido, N-amido, amino and NRₐR_{b} as defined above.

A “hydroxy” group refers to an —OH group.

An “azido” group refers to a —N═N group.

An “alkoxy” group refers to both an —O-alkyl and an —O-cycloalkyl group, as defined herein.

An “aryloxy” group refers to both an —O-aryl and an —O-heteroaryl group, as defined herein.

A “thiohydroxy” group refers to a —SH group.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “carbonyl” group refers to a —C(═O)—R″ group, where R″ is hydrogen, alkyl, cycloalkyl, aryl, heteroaryl (bonded through a ring carbon) or heteroalicyclic (bonded through a ring carbon) as defined herein.

An “aldehyde” group refers to a carbonyl group, where R″ is hydrogen.

A “thiocarbonyl” group refers to a —C(═S)—R″ group, where R″ is as defined herein.

A “C-carboxy” group refers to a —C(═O)—O—R″ groups, where R″ is as defined herein.

An “O-carboxy” group refers to an R″C(═O)—O— group, where R″ is as defined herein.

A “carboxylic acid” group refers to a C-carboxyl group in which R″ is hydrogen.

A “halo” group refers to fluorine, chlorine, bromine or iodine.

A “trihalomethyl” group refers to a —CX group wherein X is a halo group as defined herein.

A “trihalomethanesulfonyl” group refers to an X₃CS(═O)₂— group wherein X is a halo group as defined herein.

A “sulfinyl” group refers to an —S(═O)—R″ group, where R″ is as defined herein.

A “sulfonyl” group refers to an —S(═O)₂—R″ group, where R″ is as defined herein.

An “S-sulfonamido” group refers to a —S(═O)₂—NRₐR_{b} group, with Rₐ and R_{b} as defined herein.

An “N-sulfonamido” group refers to an RₐS(═O)₂—NR_{b} group, where Rₐ and R_{b} are as defined herein.

A “trihalomethanesulfonamido” group refers to an X₃CS(═O)₂NRₐ— group, where Rₐ and X are as defined herein.

An “O-carbamyl” group refers to an —OC(═O)—NRₐR_{b} group, where Rₐ and R_{b} are as defined herein.

An “N-carbamyl” group refers to an R_{b}OC(═O)—NRₐ— group, where Rₐ and R_{b} are as defined herein.

An “O-thiocarbamyl” group refers to an —OC(═S)—NRₐR_{b} group, where Rₐ and R_{b} are as defined herein.

An “N-thiocarbamyl” group refers to an R_{b}OC(═S)NRₐ— group, where Rₐ and R_{b} are as defined herein.

An “amino” group refers to an —NH₂ group.

A “C-amido” group refers to a —C(═O)—NRₐR_{b} group, where Rₐ and R_{b} are as defined herein.

An “N-amido” group refers to an R_{b}C(═O)—NRₐ group, where Rₐ and R_{b} are as defined herein.

A “quaternary ammonium” group refers to an —NHRₐR_{b} group, wherein Rₐ and R_{b} are independently alkyl, cycloalkyl, aryl or heteroaryl.

An “ureido” group refers to an —NRₐC(═O)—NR_{b}R_{c} group, where Rₐ and R_{b} are as defined herein and R_{c} is defined as either Rₐ or R_{b}.

A “guanidino” group refers to an —RₐNC(═N)—NR_{b}R_{c} group, where Rₐ, R_{b} and R_{c} are as defined herein.

A “guanyl” group refers to an RₐR_{b}NC(═N)— group, where Rₐ and R_{b} are as defined herein.

A “nitro” group refers to an —NO₂ group.

A “cyano” group refers to a —C≡N group.

A “silyl” group refers to a —Si (R″)₃, where R″ is as defined herein.

As stated above, an indicator solution useful in implementing the present invention includes at least one indicator that is either or both an aldehyde and a compound configured to form an aromatic disubstituted aminium ion upon reaction with the uronium salt.

One family of compounds useful as indicators for implementing the teachings of the present invention are aldehydes of the structure R—CHO wherein R is selected from the group consisting of alkyl, hydroxyalkyl, trihaloalkyl, cycloalkyl, alkenyl, alkynyl, aryl, benzyl, heteroaryl, heteroalicyclic, aminobenzyl, alkylaminobenzyl, dialkylaminobenzyl, dialkoxyaminobenzyl, cinnamyl, aminocinnamyl, alkyaminocinnamyl, dialkyaminocinnamyl, alkoxyaminocinnamyl, dialkoxyaminocinnamyl, nitrobenzyl, alkylbenzyl, alkoxybenzyl, phenol, dihydroxyphenyl, trihydroxyphenyl, nitrohydroxyphenyl, hydroxycinnamyl and acetamidophenyl.

Specific aldehydes suitable for use as indicators of the present invention include, but are not limited to o-anisaldehyde, m-anisaldehyde, p-anisaldehyde, 4-acetoxybenzaldehyde, 4-acetamido benzaldehyde, 2-allyloxy benzaldehyde, 4-acetoxy-3,5-dimethoxybenz aldehyde, 4-acetoxy-3-methoxy cinnamaldehyde, α-amyl cinnamaldehyde, 9-anthraldehyde, o-aminobenzoic acid, m-aminobenzoic acid, p-aminobenzoic acid, benzaldehyde, 2-bromobenzaldehyde, 3-bromobenz aldehyde, 4-bromobenz aldehyde, 3-bromo-4-fluorobenz aldehyde, 4-bromo-2-fluorobenz aldehyde, 5-bromosalicyl aldehyde, 2-bromo-3-hydroxy-4-methoxybenz aldehyde, 5-bromo-2-hydroxy-3-methoxybenzaldehyde, 5-bromovanillin, 3-bromo-p-anisaldehyde, 5-bromo-o-anisaldehyde, 5-bromo-3-nitrosalicylaldehyde, 2,5-bis(trifluoromethyl) benzaldehyde, 3,5-bis(trifluoromethyl)benzaldehyde, α-bromocinnamaldehyde, 5-bromo-2,4-dimethoxybenzaldehyde, 5-bromoveratraldehyde, 6-bromoveratraldehyde, benzyloxy acetaldehyde, 4-butoxybenzaldehyde, 3-benzyloxybenzaldehyde, 4-benzyloxy benzaldehyde, 3-(4-methoxyphenoxy)benzaldehyde, 2-benzyloxy-3-methoxy benzaldehyde, 3-benzyloxy-4-methoxybenzaldehyde, 4-benzyloxy-3-methoxy benzaldehyde, 4-biphenylcarboxaldehyde, benzene-1,4-dicarbaldehyde, 2-benzene-1,4-dicarbaldehyde, 2-chlorobenzaldehyde, 3-chlorobenzaldehyde, 4-chlorobenzaldehyde, 2-cyanobenzaldehyde, 3-cyanobenzaldehyde, 4-cyanobenzaldehyde, 2-carboxybenzaldehyde, 3-carboxybenzaldehyde, 4-carboxybenzaldehyde, 2-chloro-4-fluorobenzaldehyde, 2-chloro-6-fluorobenzaldehyde, 3-chloro-4-fluorobenzaldehyde, 2-chloro-5-nitrobenzaldehyde, 2-chloro-6-nitrobenzaldehyde, 4-chloro-3-nitrobenzaldehyde, 5-chlorosalicylaldehyde, 5-chloro-2-nitrobenzaldehyde, cinnamaldehyde, α-chlorocinnamaldehyde, chromone-3-carboxaldehyde, cinnamaldehyde, 4-carboxybenzaldehyde, o-dimethylamino benzaldehyde, o-diethylamino benzaldehyde, o-dimethylamino cinnamaldehyde, o-diethylamino cinnamaldehyde, m-dimethylamino benzaldehyde, m-diethylamino benzaldehyde, m-dimethylamino cinnamaldehyde, m-diethylamino cinnamaldehyde, p-dimethylamino benzaldehyde, p-diethylamino benzaldehyde, p-dimethylamino cinnamaldehyde, p-diethylamino cinnamaldehyde, 2-(difluoromethoxy) benzaldehyde, 4-(difluoromethoxy) benzaldehyde, 2,3-dichlorobenzaldehyde, 2,4-dichlorobenzaldehyde, 2,6-dichlorobenzaldehyde, 3,4-dichlorobenzaldehyde, 3,5-dichlorobenzaldehyde, 2,3-difluorobenzaldehyde, 2,4-difluorobenzaldehyde, 2,5-difluorobenzaldehyde, 2,6-difluorobenzaldehyde, 3,4-difluorobenzaldehyde, 3,5-difluorobenzaldehyde, 2,3-dihydroxybenzaldehyde, 2,4-dihydroxybenzaldehyde, 2,5-dihydroxybenzaldehyde, 3,4-dihydroxybenzaldehyde, 3,5-dihydroxybenzaldehyde, 2,4-dinitrobenzaldehyde, 2,6-dinitrobenzaldehyde, 3,5-dinitrobenzaldehyde, 3,5-dibromosalicylaldehyde, 3,5-dibromo-4-hydroxy benzaldehyde, 3,5-dichloro salicylaldehyde, 3,5-diiodo salicylaldehyde, 3,4-dihydroxy-5-methoxy benzaldehyde, 2,6-dimethoxy-4-hydroxy benzaldehyde, 3,4-dimethoxy-5-hydroxy benzaldehyde, 4,6-dimethoxy salicylaldehyde, 2,3-dimethoxy benzaldehyde, 2,4-dimethoxybenzaldehyde, 2,5-dimethoxybenzaldehyde, 2,6-dimethoxybenzaldehyde, 3,5-dimethoxybenzaldehyde, 2,4-dimethylbenzaldehyde, 2,5-dimethylbenzaldehyde, 3,5-dimethylhydroxylbezaldehyde, 2,3-dimethyl-p-anisaldehyde, 2,5-dimethyl-p-anisaldehyde, 2,4-dimethoxy-3-methylbenzaldehyde, 4-(diethylamino) salicylaldehyde, diphenylacetaldehyde, 3,4-dihydroxybenzaldehyde, 3,5-dimethoxy-4-hydroxy-benzaldehyde, 2,4-dimethoxybenzaldehyde, 2,3-dimethoxybenzaldehyde, 2,5-dimethoxybenzaldehyde, 3,5-dimethoxybenzaldehyde, 3,4-dimethoxybenzaldehyde, 4-dimethylamino-1-naphthaldehyde, 4-dimethylamino-2-methoxybenzaldehyde, 2,4-dihydroxybenzaldehyde, 3,4-dihydroxybenzaldehyde, 2,5-dihydroxybenzaldehyde, 4-dibutylamino-benzaldehyde, 4-diethylamino-2-hydroxybenzaldehyde, 3,5-dimethoxy-4-hydroxy-cinnamaldehyde, 3-methoxy-4-(1-pyrrolidinyl)-benzaldehyde, 4-diethylamino-3-methoxybenzaldehyde, 3,5-dimethyl-4-hydroxybenzaldehyde, 3,5-di-tert-butyl-2-hydroxy benzaldehyde, 3,5-di-tert-butyl-4-hydroxybenzaldehyde, 3,4-dimethoxy-5-hydroxy benzaldehyde, 5-(4-(diethylamino) phenyl)-2,4-pentadienal, 3,4-dihydroxy-benzaldehyde, 3,5-dimethoxy-4-hydroxy-benzaldehyde, 3,5,-dimethyl-4-hydroxybenzaldehyde, 4-dimethylamino-2-methoxy benzaldehyde, 4-dimethylamino-1-naphthaldehyde, 2,3-dimethoxy-benzaldehyde, 2,5-dimethoxybenzaldehyde, 3,5-dimethoxy-benzaldehyde, 3,4-dimethoxy benzaldehyde, 4-dibutylamino-benzaldehyde, 4-diethylamino-2-hydroxy benzaldehyde, 3,4-dimethoxy-5-hydroxy-benzaldehyde, 5-(4-(diethylamino)phenyl)-2,4-pentadienal, 2,4-dihydroxybenzaldehyde, 4-dihydroxy-benzaldehyde, 3,4-dihydroxy-benzaldehyde, 2,5-dihydroxybenzaldehyde, 2,4-dimethoxybenzaldehyde, 3,5-dimethoxy-4-hydroxy-cinnamaldehyde, 4-diethylamino-3-methoxybenzaldehyde, 2-ethoxybenzaldehyde, 4-ethoxybenzaldehyde, 3-ethoxy-4-hydroxybenzaldehyde, 3-ethoxysalicylaldehyde, 3-ethoxy-4-hydroxy benzaldehyde, N-ethylcarbazole-3-aldehyde, 3-ethoxy-4-methoxy benzaldehyde, 4-ethoxy benzaldehyde, 4-ethoxy benzaldehyde, 3-ethoxy-4-hydroxy benzaldehyde, 2-fluoro benzaldehyde, 3-fluorobenzaldehyde, 4-fluorobenzaldehyde, 2-formylphenylboronic acid, 3-formylphenylboronic acid, 4-formylphenylboronic acid, 2-formylbenzenesulfonic acid, 2-fluoro-5-nitrobenzaldehyde, 3-fluorosalicylaldehyde, 4-formyl-1,3-benzenedisulfonic acid, 2-fluoro-3-(trifluoromethyl)benzaldehyde, 2-fluoro-6-(trifluoromethyl)benzaldehyde, 4-fluoro-2-(trifluoromethyl) benzaldehyde, 4-fluoro-3-(trifluoromethyl)benzaldehyde, 3-fluoro-p-anisaldehyde, 3-fluoro-2-methlbenzaldehyde, 4-ethylbenzaldehyde, 2-fluorenecarboxaldehyde, 3-hydroxybenzaldehyde, 4-hydroxy benzaldehyde, 2-hydroxy-5-nitrobenzaldehyde, 3-hydroxy-4-nitro benzaldehyde, 4-hydroxy-3-nitro benzaldehyde, 5-hydroxy-2-nitrobenzaldehyde, 2-hydroxy-4-methoxybenzaldehyde, 2-hydroxy-5-methoxybenzaldehyde, 3-hydroxy-4-methoxybenzaldehyde, 2-hydroxy-5-methyl-1,3-benzenedicarboxaldehyde, hydrocinnamaldehyde, 2-hydroxy-1-naphthaldehyde, 4-(hexyloxy)benzaldehyde, 4-hydroxy-3-methoxybenzaldehyde (vanillin), 3-hydroxy-4-methoxy-benzaldehyde (isovanillin), 4-hydroxybenzaldehyde, 4-hydroxy-2-methoxybenzaldehyde, 2-hydroxybenzaldehyde, 4-hydroxy-1-naphthaldehyde, 4-methoxy-1-naphthaldehyde, 4′-hydroxy-biphenyl-1-carbaldehyde, 2-hydroxy-3-methoxybenzaldehyde, 4-hydroxy-3-methoxy-cinnamaldehyde, 6-hydroxychromen-3-carbox-aldehyde, α-hexylcinnamaldehyde, 4-hydroxy-3-methoxy-cinnamaldehyde, 4-hydroxy-benzaldehyde, 4-hydroxy-2-methoxy-benzaldehyde, 2-hydroxybenzaldehyde, 4-hydroxy-1-naphthaldehyde, 4′-hydroxy-biphenyl-1-carbaldehyde, 2-hydroxy-3-methoxybenzaldehyde, isophthalaldehyde, 5-iodovanillin, 4-isopropylbenzaldehyde, indole-3-carbaldehyde, 6-methyl-2-pyridinecarboxaldehyde, 2,3-(methylenedioxy) benzaldehyde, 3-methoxy-5-nitrosalicylaldehyde, 3-methyl-p-anisaldehyde, 2-methoxycinnamaldehyde, mesitaldehyde, 2-methoxy-1-naphthaldehyde, N-methylpyrrole-2-aldehyde, 5-methylfurfural, 6-methylindole-3-carboxaldehyde, 6-methyl-4-oxo-1 (4H)-benzopyran-3-carbaldehyde, 2-methyl-1,4-naphthoquinone, 4-carboxybenzaldehyde, 4-methoxy-1-naphthalaldehyde, methyl 2-formyl-3,5-dimethoxybenzoate, 2-methoxy-1-naphthalaldehyde, 4-methyl-5-imidazole-carboxaldehyde, 2-methyl-1,4-naphthoquinone, 3-methoxy-4-(1-pyrrolidinyl)-benzaldehyde, 2-methoxy-1-naphthaldehyde, 4-methoxy-1-naphthaldehyde, 2-nitrobenzaldehyde, 3-nitrobenzaldehyde, 4-nitrobenzaldehyde, 5-nitro-vanillin, 2-nitrocinnamaldehyde, 4-nitrocinnamaldehyde, 2-nitrobenzaldehyde, 3-nitrobenzaldehyde, 4-nitrobenzaldehyde. 6-nitroveratraldehyde, 1-naphthaldehyde, 2-naphthaldehyde, 2,3-naphthalendicarboxaldehyde, 1,8-naphthalaldehydic acid, 2-nitrobenzaldehyde, 3-nitrobenzaldehyde, 4-nitrobenzaldehyde, phenylacetaldehyde, 2-pyridinecarboxaldehyde, 3-pyridinecarboxaldehyde, 4-pyridinecarboxaldehyde, 4-pyridinecarboxaldehyde N-oxide, phthalaldehyde, piperonal, 2-phenyl propionaldehyde, pentafluorobenzaldehyde, 3-phenylbutyraldehyde, 4-propoxybenzaldehyde, pentamethylbenzaldehyde, 3-phenoxybenzaldehyde, 4-phenoxybenzaldehyde, phenylpropargyl aldehyde, 1,2-phthaldialdehyde, pyrrole-2-aldehyde, phthalimidoacetaldehyde, o-phtalaldehyde, 1,2-phthaldialdehyde, 2-quinolinecarboxaldehyde, 3-quinolinecarboxaldehyde, 4-quinolinecarboxaldehyde, salicylaldehyde, syringaldeyde, o-tolualdehyde, m-tolualdehyde, p-tolualdehyde, 3-(trifluoromethoxy)benzaldehyde, 4-(trifluoromethoxy)benzaldehyde α,α,α-trifluoro-o-tolualdehyde α,α,α-trifluoro-m-tolualdehyde, α,α,α-trifluoro-p-tolualdehyde, terephthalaldehyde, 5-(trifluoromethoxy)salicylaldehyde, 2,3,5-trichloro benzaldehyde, 2,3,6-trichlorobenzaldehyde, 2,3,4-trifluorobenzaldehyde, 2,3,6-trifluoro benzaldehyde, 2,3,4-trihydroxy benzaldehyde, 2,4,6-trihydroxybenzaldehyde, 3,4,5-trihydroxy benzaldehyde, 2,3,5,6-tetrafluorobenzaldehyde, 2,3,4-trimethoxy benzaldehyde, 2,4,5-trimethoxybenzaldehyde, 2,4,6-trimethoxybenzaldehyde, 3,4,5-trimethoxy benzaldehyde, 4-4-tert-butylbenzaldehyde, 3-tert-butyl-2-hydroxy benzaldehyde, 5-tert-butyl-2-hydroxy benzaldehyde, 2,3,4-trihydroxy benzaldehyde, 3,4,5-trihydroxybenzaldehyde, 2,3-thiophene-dicarboxaldehyde, 2,5-thiophene-dicarboxaldehyde, thiophene-2-aldehyde, thiophene-3-aldehyde, 2,4,6-trihydroxy benzaldehyde, 2,3,4-trihydroxybenzaldehyde, 3,4,5-trihydroxybenzaldehyde, 2,4,6-trihydroxy benzaldehyde, vanillin, o-vanillin, vinyl benzaldehyde, veratraldehyde and vanillin acetate.

In a preferred embodiment of the present invention, the indicator is an aromatic aldehyde of the structure Ar—(CR_{n,a}═CR_{n,b})ₙ—CH═O where: n is an integer 0 or greater; Ar is aromatic; and each one of R_{n,a} and R_{n,b} are independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl, cyano, nitro, amino, —NR₁₅R₁₆, where R₁₅ and R₁₆ are independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl and cyano, or, alternatively, at least two of substituents of Ar, R₁₅, R₁₆, R_{n,a} and/or R_{n,b} form an aromatic, heteroaromatic, alicyclic or heteroalicyclic ring. According to a feature of the present invention, the aromatic group Ar is substituted with at least one positive charge stabilizing functional group, preferably para to the —(CR_{n,a}═CR_{n,b})ₙ—CH═O group. Preferred positive charge stabilizing functional groups include but are not limited to amines, alkylamines, dialkylamines, alcohols, esters, amides, acids and alkyls. In the currently known best mode of the present invention, at least one of the at least one positive charge stabilizing functional groups is a disubstituted amine, preferably a dialkyl amine, preferably para to the —(CR_{n,a}═CR_{n,b})ₙ—CH═O group. Suitable aromatic aldehydes include but are not limited to p-DMAC, p-DMAB, p-DEAC and p-DEAB.

In a preferred embodiment of the method of the present invention, the presence of an uronium salt in a sample is determined by forming an aromatic disubstituted aminium ion from an indicator solution, the disubstituted aminium ion of the structure: 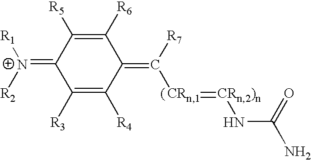
wherein n is 0 or greater; and
wherein R₁, R₂, R₃, R₄, R₅, R₆, R₇, R_{n,1} and R_{n,2} are each independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl, cyano, nitro, amino and —NR₈R₉, R₈ and R₉ independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl and cyano, or alternatively, at least two of R₁, R₂, R₃, R₄, R₅, R₆, R₇, R₈, R₉, R_{n,1} and R_{n,2} are part of an aromatic, heteroaromatic, alicyclic or heteroalicyclic ring. Preferably, the aromatic disubstituted aminium ion is an aromatic dialkylaminium ion, R₁ and R₂ each being independently selected from the group consisting of methyl and alkyl.

Generally, useful as indicators in implementing the present invention are compounds that react in a manner analogous to that described in the introduction for p-DMAC and generally include aromatic acids, amides, ketones and aldehydes that are susceptible to nucleophilic attack by an amine on a protonated carbonyl group to form an imine. Further, a resulting imine preferably rearranges to a conjugated system so as to significantly change color, absorbance or fluorescence subsequent to reaction. Preferred compounds useful as indicators for implementing the teachings of the present invention and for forming an aromatic disubstituted aminium ion include aldehydes, ketones, acids, amides and the like of the structure: 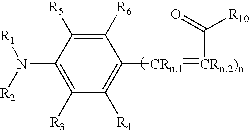
wherein n is 0 or greater; and
wherein R₁, R₂, R₃, R₄, R₅, R₆, R₁₀, R_{n,1} and R_{n,2} are each independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl, cyano, nitro, amino and —NR₈R₉, R₈ and R₉ independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl and cyano, or alternatively, at least two of R₁, R₂, R₃, R₄, R₅, R₆, R₈, R₉, R₁₀, R_{n,1} and R_{n,2} are part of an aromatic, heteroaromatic, alicyclic or heteroalicyclic ring.

To increase the relative concentration of aminium ions at the expense of imine ions and thus to increase the sensitivity of the method of the present invention it is preferred, for example, that R₁ and R₂ are each independently selected from the group consisting of methyl and alkyl or alternatively, at least one of R₁ or R₂ is part of an aromatic, heteroaromatic, alicyclic or heteroalicyclic ring, to give a dialkyl aminium ion. Families of compounds that fulfill these preferences and this are exceptionally useful as indicators for implementing the present invention include, but are not limited to p-dialkylaminocinnamaldehydes, p-dialkylaminocinnamyl ketones, linear p-dialkylaminocinnamic acids, cyclic p-dialkylaminocinnamic acids (coumarins), linear p-dialkylaminocinnamamides, cyclic p-dialkylaminocinnamamides (quinolinones), p-dialkylaminobenzaldehydes, p-dialkylaminophenyl ketones, p-dialkylaminobenzoic acids, p-dialkylamino benzamides, salts thereof and esters thereof.

As used herein, the term “derivative” describes the result of a chemically altering, modifying or changing a molecule or a portion thereof, such that it maintains its original functionality in at least one respect.

p-dialkylaminocinnamaldehydes have the structure: 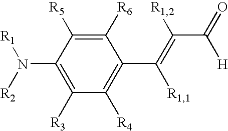
and include such compounds as p-dimethylaminocinnamaldehyde (p-DMAC) and p-diethylaminocinnamaldehyde (p-DEAC).

p-dialkylaminocinnamyl ketones have the structure: 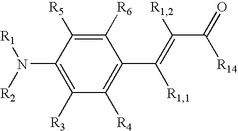
and include such compounds as (3E)-4-[4-(dimethylamino)phenyl]-3-buten-2-one, 1E)-1-[4-(dimethylamino)phenyl]-4,4-dimethyl-1-penten-3-one, (2E)-3-[4-(dimethylamino)phenyl]-1-(2-furyl)-2-propen-1-one, (2E)-3-[4-(dimethylamino)phenyl]-1-phenyl-2-propen-1-one, 3-[4-(dimethylamino)phenyl]-1-(4-methylphenyl)-2-propen-1-one, (3E)-4-[4-(diethylamino)phenyl]-3-buten-2-one, (1E)-1-[4-(diethylamino)phenyl]-4,4-dimethyl-1-penten-3-one, (2E)-3-[4-(diethylamino)phenyl]-1-(2-furyl)-2-propen-1-one, (2E)-3-[4-(diethylamino)phenyl]-1-phenyl-2-propen-1-one and 3-[4-(diethylamino)phenyl]-1-(4-methylphenyl)-2-propen-1-one.

Linear p-dialkylaminocinnamic acids have the structure: 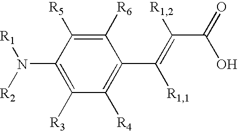
and include such compounds as 3-(4-amino-2-methylphenyl) acrylic acid, 4-(dimethylamino)cinnamic acid (CAS 1552-96-1), (2E)-3-[4-(dimethylamino)phenyl]-2-methyl-2-propenoic acid, (2E)-2-cyano-3-[4-(dimethylamino)phenyl]-2-propenoic acid and ethyl 2-cyano-3-[4-(dimethylamino)phenyl]acrylate, salts thereof or esters thereof.

Cyclic p-dialkylaminocinnamic acids (coumarins) have the structure: 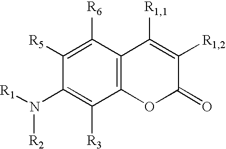
and include such compounds as coumarin 110 (CAS 20571-42-0), coumarin 6H (CAS 58336-35-9), 3-acetyl-7-(diethylamino)-2H-chromen-2-one, 7-amino-4-methylcoumarin, 7-(diethylamino)coumarin-3,4-dicarboxylic acid (CAS 75240-77-6), 1-[7-(diethylamino)-3-coumarinylcarbonyl]imidazole (CAS 261943-47-9), N-succinimidyl 7-(diethylamino)coumarin-3-carboxylate (CAS 139346-57-9), 7-(diethylamino)coumarin-3-carboxylic acid (CAS 50995-74-9), 7-(diethylamino)coumarin-3-carbonyl azide (CAS 157673-16-0), 7-(diethylamino)coumarin-3-carbohydrazide (CAS 100343-98-4), 7-(diethylamino)coumarin (CAS 20571-42-0), 3-(2-N-methylbenzimidazolyl)-7-N,N-diethylaminocoumarin (CAS 41044-12-6), 3-(2-benzothiazolyl)-7-(diethylamino)coumarin (CAS 38215-36-0), N-succinimidyl 3-(2-benzothiazolyl)-7-(diethylamino)coumarin-4-carboxylate (CAS none) and 3-(2-benzothiazolyl)-7-(diethylamino)coumarin-4-carboxylic acid (CAS 136997-14-3).

Cyclic p-dialkylaminocinnamides (quinolinones) have the structure: 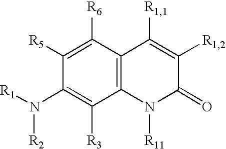
where R₁₁ is selected from the group consisting of H, methyl, alkyl, hydroxyalkyl, trihaloalkyl, cycloalkyl, alkenyl, alkynyl, aryl, benzyl, heteroaryl, heteroalicyclic and halo and include such compounds as 7-(dimethylamino)-4-methyl-2(1H)-quinolinone (CAS 26078-23-9) and 7-(dimethylamino)-2(1H)-quinolinone.

Linear p-dialkylaminocinnamamides have the structure: 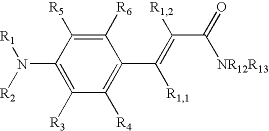
where R₁₂ and R₁₃ are each independently selected from the group consisting of H, methyl, alkyl, hydroxyalkyl, trihaloalkyl, cycloalkyl, alkenyl, alkynyl, aryl, benzyl, heteroaryl, heteroalicyclic and halo and include such compounds as 2-cyano-3-[4-(dimethylamino)phenyl]acrylamide and (2E)-2-cyano-3-[4-(diethylamino)phenyl]-N-methyl-2-propenamide.

p-dialkylaminobenzaldehydes have the structure: 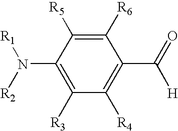
and include such compounds as p-dimethylaminobenzaldehyde (p-DMAB) and p-diethylaminobenzaldehyde (p-DEAB).

p-dialkylaminophenyl ketones have the structure: 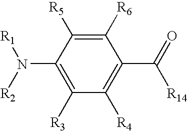
and include such compounds as p-dimethylaminophenyl methyl ketone, p-dimethylaminophenyl ethyl ketone, p-dimethylaminophenyl phenyl ketone, p-diethylaminophenyl methyl ketone, p-diethylaminophenyl ethyl ketone, p-diethylaminophenyl phenyl ketone, p-dimethylamino acetophenone 4′-piperidinoacetophenone (CAS 10342-85-5), 4′-piperazinoacetophenone (CAS 51639-48-6), 4′-morpholinoacetophenone (CAS 39910-98-0), 4′-(dimethylamino)-2,2,2-trifluoroacetophenone (CAS 2396-05-6), 1-[4-(4-hydroxy-1-piperidinyl)phenyl]ethanone, 1-[4-(4-morpholinyl)phenyl]-1-propanone and 4-(dimethylamino)phenyl](phenyl)methanone (CAS 530-44-9).

p-dialkylaminobenzoic acids have the structure: 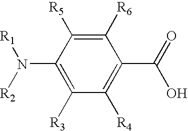
and include such compounds as methyl 4-(diphenylamino) benzoate, 4-(dimethylamino) benzoic acid (CAS 619-84-1), 4-(diethylamino) benzoic acid (CAS 5429-28-7), salts thereof or esters thereof.

p-dialkylaminobenzamides have the structure: 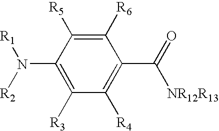
wherein R₁₂ and R₁₃ are each independently selected from the group consisting of H, methyl, alkyl, hydroxyalkyl, trihaloalkyl, cycloalkyl, alkenyl, alkynyl, aryl, benzyl, heteroaryl, heteroalicyclic and halo and include such compounds as p-dimethylaminobenzamide (p-DMAB) and p-diethylaminobenzamide (p-DEAB).

The method of the present invention for determining the presence or absence of an uronium salt in a sample is based on contacting the sample with an indicator in an indicator solution having a pH greater than about 2, the indicator selected as discussed above. An appropriate change in the appearance of the indicator solution subsequent to the contacting with the sample indicates the presence of an uronium salt. Appropriate changes in the appearance of the indicator solution include a change in color, a change in light absorption and a change in fluorescence of the solution.

For the required chromogenic reaction to occur it is generally necessary that the indicator solution include at least one protic solvent. Suitable protic solvents include but are not limited to water, alcohol, ethanol, methanol, propanol, isopropanol, butanol, isoamylalcohol, glycol and 1,2-dihydroxypropane.

It is often advantageous to include, in addition to the at least one protic solvent at least one aprotic solvent. Suitable aprotic solvents include but are not limited to ethers, acetates, ketones, acetone, butyl acetate, acetonitrile, chloroform, dichloromethane, diethyl ether, dimethyl formamide, dimethylsulfoxide, ethyl acetate and tetrahydrofuran.

In a preferred embodiment, a protic solvent (preferably water) is combined with at least one second, volatile, solvent (protic or aprotic) and used in an indicator solution of the present invention. When a sample is contacted with such an indicator solution, the volatile solvent is allowed to evaporate. If the sample does not contain an uronium salt the pH does not change as the volatile solvent evaporates. However, if the sample does contain an uronium salt, the pH becomes increasingly acidic and the concentration of sample in the solution increases as the volatile solvent evaporates. Using such a combination of solvents, the sensitivity of the method of the present invention is increased. One volatile/non-volatile solvent combination useful in implementing the method of the present invention is a mixture of ethanol and water.

Generally, the preferred concentration of an indicator in an indicator solution is not critical to the practice of the teachings of the present invention. That said, the concentration of the indicator in the indicator solution is preferably less than about 4% by weight, less than about 1% by weight and even less than about 0.5% by weight, but generally greater than about 0.1% by weight.

Generally, implementation of the teachings of the present invention requires dispensing an indicator solution of the present invention from a vessel where the indicator solution is held. Suitable vessels include but are not limited to aerosol dispensers, bags, beakers, bottles, droppers, jars, mechanically actuated spray dispensers, pressurized spray dispensers, pump spray dispensers, sacks, sachets, spray dispensers, squeeze bottles, ampoules, syringes, capsules, foil wrappers and tubes.

In a preferred embodiment of the present invention, a sample is collected and/or concentrated for testing.

In one embodiment, the collecting or concentrating is performed prior to contacting the sample with the indicator solution. For example, in one embodiment of the present invention, by collecting or concentrating is meant gathering the sample from an area, for example by wiping an area where the sample is found with a collector and/or concentrator. Suitable collectors and/or concentrators include but are not limited to bibulous materials, cloth, fabrics, felt, flannel, membranes, pads, papers, sponges, swabs, swatches, tissues and wipes.

According to a feature of the present invention, the contacting of the sample with the indicator solution occurs on the collector and/or the concentrator.

In one embodiment, the collector and/or the concentrator is impregnated with the indicator solution prior to the wiping, e.g., a “wet-wipe”. In a “wet-wipe” implementation, a pad impregnated with an indicator solution of the present invention is stored in, for example, a foil packet. When it is desired to test an area suspected of having an uronium salt, the foil packet is torn open, the pad removed therefrom and wiped across the surface. The action of wiping gathers sample from a large area, collecting and concentrating the sample. If an uronium salt is present, an appropriate change in the appearance of the pad is observed.

In another embodiment, the indicator is applied onto the collector and/or the concentrator prior to the wiping. For example, when it is desired to test an area suspected of having an uronium salt, a tissue is dipped in a bottle containing an indicator solution of the present invention. The tissue is then wiped across the surface. The action of wiping gathers sample from a large area, collecting and/or concentrating the sample. If an uronium salt is present, a change in the appearance of the tissue is observed.

In an even further embodiment, the indicator is applied onto the collector and/or the concentrator subsequent to the wiping. For example, when it is desired to test an object suspected as having been in contact with an uronium salt, a swab is wiped on the object. The action of wiping gathers sample from a large area, collecting and/or concentrating the sample. An indicator solution of the present invention is then applied to the swab, for example by spraying or dripping. If an uronium salt is present, a change in the appearance of the swab is observed.

In an even further embodiment, the contacting of the sample with the indicator solution occurs on a location where the sample is found, by applying the indicator solution to a location where the sample is found. For instance, an indicator solution of the present invention is sprayed onto an area suspected of having an uronium salt. A change in the appearance of the sprayed area is observed if an uronium salt is present.

In the art the kits and devices for implementing colorimetric detection methods is known, see for example, U.S. Pat. No. 5,296,380, U.S. Pat. No. 5,457,054 and U.S. Pat. No. 5,648,047. Useful in implementing the method of the present invention is a kit of the present invention.

A kit of the present invention for determining the presence or absence of an uronium salt in a sample generally includes: a) at least one indicator as described hereinabove; b) a device selected from amongst a collector, a concentrator or a combination thereof, and c) a vessel configured to hold and dispense a solution of the indicator having a pH of greater than about 2. Preferably, the indicator is packaged in a packaging material and identified in print, in or on the packaging material, for use for determining the presence or absence of an uronium salt in a sample as well as instructions for use, safety data and the like.

As certain explosives may contain urea nitrate or other uronium salts, a kit of the present invention for determining the presence or absence of an explosive in a sample (e.g. wherein the explosive comprises an uronium salt such as urea nitrate): generally includes: a) at least one indicator as described hereinabove; b) a device selected from amongst a collector, a concentrator or a combination thereof, and c) a vessel configured to hold and dispense a solution of the indicator having a pH of greater than about 2. Preferably, the indicator is packaged in a packaging material and identified in print, in or on the packaging material, for use for determining the presence or absence of an explosive in a sample as well as instructions for use, safety data and the like.

In a kit of the present invention, the indicator is provided in a concentrated form or as a ready-to-use indicator solution.

Generally the concentrated form of an indicator in a kit of the present invention is configured so as to reduce volume and weight of a respective kit, but also allow for quick preparation of a solution of the present invention. Quick preparation includes such factors as ease of dissolution and distribution into appropriately sized portions. When it is desired to prepare an indicator solution of the present invention from a concentrated form, an appropriate amount or a portion is taken and combined with an appropriate amount of solvent. Preferred concentrated forms include a substantially pure indicator in solid or liquid form, or a solid or liquid containing indicator. Generally high percentages of the indicator is preferred, e.g. greater than about 1% by weight, greater than about 5% by weight, greater than about 10% by weight, greater than about 20% by weight, greater than about 30% by weight, greater than about 50% by weight or even greater than about 80% by weight of indicator. If a solid concentrated form is used, the solid concentrated form is preferably divided into portions and provided as tablets, capsules, powder in a bag and the like. If a liquid concentrated form is used, the liquid concentrated form is preferably divided into portions and contained within an ampoule, capsule, syringe and the like, or packaged in a dispensing type package configured to dispense an appropriate portion such as a package equipped with a dropper, a syringe, a pump and the like.

In a preferred embodiment, a portion of concentrated indicator is such that when combined with an amount of solvent that fits in the vessel for holding and dispensing the indicator solution, an appropriate solution in terms of pH, concentration and other parameters, is made.

When the indicator is provided in concentrated form in a kit of the present invention, it is preferable to also provide a solvent as part of the kit. Preferably, the type and amount of solvent provided is chosen so that upon mixing with the concentrated form of the indicator, an indicator solution with an appropriate pH is made.

When the indicator is provided as a ready-to-use indicator solution in a kit of the present invention, the indicator solution is preferably as described hereinabove.

A kit of the present invention is preferably provided with a device (or devices) that is a collector and/or a concentrator. Collectors and/or concentrators useful for implementing a kit of the present invention include but are not limited to bibulous materials, cloth, fabrics, felt, flannel, membranes, pads, papers, sponges, swabs, swatches, tissues and wipes. In a preferred embodiment of a kit of the present invention, a collector and/or a concentrator provided with a kit of the present invention is impregnated with the indicator, whether as a ready-to-use solution or in a concentrated form.

A kit of the present invention is preferably provided with a vessel configured to hold and dispense an indicator solution of the present invention. Preferred vessels include but are not limited to aerosol dispensers, bags, beakers, bottles, droppers, jars, mechanically actuated spray dispensers, pressurized spray dispensers, pump spray dispensers, sacks, sachet, spray dispensers, squeeze bottles, ampoules, syringes, capsules, foil wrappers and tubes.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above description, illustrate the invention in a non-limiting fashion.

### Materials and Experimental Methods

p-Dimethylaminocinnamaldehyde (p-DMAC), p-dimethylaminobenzaldehyde (p-DMAB) and ethanol are commercially available and were purchased from Sigma-Aldrich (St. Louis, Mo., USA). Urea nitrate was prepared in the usual way.

Solution I was prepared by dissolving 4 gram of p-DMAC in 1 liter of 96% ethanol (with water) to yield a 0.4% p-DMAC solution. A drop of solution I was applied to litmus paper and the resulting color indicated a pH of 7. Subsequent to the application of a drop of water to the location where solution I had been previously applied the resulting color indicated a pH between 7 and 8.

Solution II was prepared by dissolving 4 gram of p-DMAB in 1 liter of 96% ethanol (with water) to yield a 0.4% p-DMAB solution. A drop of solution II was applied to litmus paper and the resulting color indicated a pH of 7. Subsequent to the application of a drop of water to the location where solution II had been previously applied the resulting color indicated a pH between 7 and 8.

A urea nitrate solution was prepared by dissolving 1 gram urea nitrate in 1 liter water. On strips of filter paper, varying amounts of the urea nitrate solution were applied to make a series of spots with known amounts of urea nitrate. Further, varying amounts of the urea nitrate solution were put in wells of a white-colored porcelain spot test plate.

### EXPERIMENTAL RESULTS

### Example 1

### Detection of Urea Nitrate Using p-DMAC Solution I

10 microliter of solution I were applied to a series of urea nitrate spots on filter paper. Within 1 minute of application, a bright red-purple color was observed with the naked eye on all spots having 10 microgram or more of urea nitrate.

10 microliter of solution I were added to a series of urea nitrate containing wells in the white colored porcelain spot test plate. Within 1 minute of addition, a bright red-purple color was observed in all wells having 1 microgram or more of urea nitrate.

### Example 2

### Lower Limit of Detection of Urea Nitrate Using p-DMAB Solution

10 microliter of solution II were applied to a series of urea nitrate spots on filter paper. Within 1 minute of application, a bright lemon yellow color was observed with the naked eye on all spots having 100 microgram or more of urea nitrate.

10 microliter of solution II were added to a series of urea nitrate containing wells in the white colored porcelain spot test plate. Within 1 minute of addition, a bright lemon yellow color was observed in all wells having 20 microgram or more of urea nitrate.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific examples thereof, it is evident that many alternatives, modifications and variations will be apparent to those skilled in the art. Accordingly, it is intended to embrace all such alternatives, modifications and variations that fall within the spirit and broad scope of the appended claims. For example, there are advantages in using a combination of two or more different indicators in one indicator solution as described above. Such a use is within the scope of the claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. Colorimetric identification of uronium salts.

2. Colorimetric detection of urea nitrate.

3. A use of an indicator for determining the presence or absence of an uronium salt in a sample, said indicator selected from the group consisting of an aldehyde and a compound configured to form an aromatic disubstituted aminium ion upon reaction with said uronium salt.

4. A use of an indicator solution for determining the presence or absence of an uronium salt in a sample, the indicator solution having a pH of greater than about 2 and comprising at least one indicator selected from the group consisting of an aldehyde and a compound configured to form an aromatic disubstituted aminium ion upon reaction with said uronium salt.

5. The use of claim 4, wherein said indicator solution has a pH of greater than about 3.

6. The use of claim 4, wherein said indicator solution has a pH of greater than about 4.

7. The use of claim 4, wherein said indicator solution has a pH of greater than about 5.

8. The use of claim 4, wherein said indicator solution has a pH of greater than about 6.

9. The use of claim 4, wherein said indicator solution has a pH of greater than about 7.

10. The use of claim 3, wherein said aldehyde is an aromatic aldehyde.

11. The use of claim 3, wherein said aromatic disubstituted aminium ion is an aromatic dialkylaminium ion.

12. The use of claim 3, wherein at least one of said at least one indicator comprises p-dimethylaminocinnamaldehyde (p-DMAC), diethylaminocinnamaldehyde (p-DEAC), p-dimethylaminobenzaldehyde (p-DMAB), p-diethylaminobenzaldehyde (p-DEAB) or a mixture thereof.

13. The uses of claim 3, wherein said uronium salt is urea nitrate.

14. A method of determining the presence of an uronium salt in a sample comprising forming an aromatic disubstituted aminium ion from an indicator solution, said indicator solution having a pH of greater than about 2, said disubstituted aminium ion of the structure: 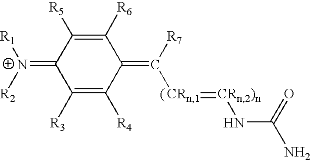
wherein n is 0 or greater and
wherein R₁, R₂, R₃, R₄, R₅, R₆, R₇, R_{n,1} and R_{n,2} are each independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl, cyano, nitro, amino and —NR₈R₉,
R₈ and R₉ independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl and cyano,
or alternatively, at least two of R₁, R₂, R₃, R₄, R₅, R₆, R₇, R₈, R₉, R_{n,1} and R_{n,2} are part of an aromatic, heteroaromatic, alicyclic or heteroalicyclic ring.

15. The method of claim 14, said indicator solution having a pH of greater than about 3.

16. The method of claim 14, said indicator solution having a pH of greater than about 4.

17. The method of claim 14, said indicator solution having a pH of greater than about 5.

18. The method of claim 14, said indicator solution having a pH of greater than about 6.

19. The method of claim 14, said indicator solution having a pH of greater than about 7.

20. The method of claim 14, wherein said aromatic disubstituted aminium ion is an aromatic dialkylaminium ion, R₁ and R₂ each being independently selected from the group consisting of methyl and alkyl.

21. A method of identifying a sample as containing an uronium salt, comprising reacting the sample with an indicator solution including at least one indicator of the structure: 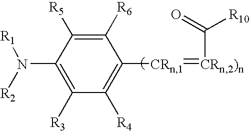
wherein n is 0 or greater and
wherein R₁, R₂, R₃, R₄, R₅, R₆, R₁₀, R_{n,1} and R_{n,2} are each independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl, cyano, nitro, amino and —NR₈R₉,
R₈ and R₉ independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl and cyano,
or alternatively, at least two of R₁, R₂, R₃, R₄, R₅, R₆, R₈, R₉, R₁₀, R_{n,1} and R_{n,2} are part of an aromatic, heteroaromatic, alicyclic or heteroalicyclic ring;
said indicator solution having a pH greater than about 2.

22. The method of claim 21, said indicator solution having a pH of greater than about 3.

23. The method of claim 21, said indicator solution having a pH of greater than about 4.

24. The method of claim 21, said indicator solution having a pH of greater than about 5.

25. The method of claim 21, said indicator solution having a pH of greater than about 6.

26. The method of claim 21, said indicator solution having a pH of greater than about 7.

27. The method of claim 21, wherein at least one of said at least one indicator is selected from the group consisting of p-dialkylaminocinnamaldehydes, p-dialkylaminocinnamyl ketones, linear p-dialkylaminocinnamic acids, cyclic p-dialkylaminocinnamic acids (coumarins), linear p-dialkylaminocinnamamides, cyclic p-dialkylaminocinnamamides (quinolinones), p-dialkylaminobenzaldehydes, p-dialkylaminophenyl ketones, p-dialkylaminobenzoic acids, p-dialkylamino benzamides, salts thereof and esters thereof.

28. The method of claim 27, wherein said indicator is a p-dialkylaminocinnamaldehyde of the structure: 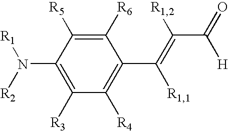
wherein R₁ and R₂ are each independently selected from the group consisting of methyl and alkyl or alternatively, at least one of R₁ or R₂ is part of an aromatic, heteroaromatic, alicyclic or heteroalicyclic ring.

29. The method of claim 27, wherein said indicator is a p-dialkylaminocinnamaldehyde selected from the group consisting of p-dimethylaminocinnamaldehyde (p-DMAC) and p-diethylaminocinnamaldehyde (p-DEAC).

30. The method of claim 27, wherein said indicator is a p-dialkylaminocinnamyl ketone of the structure: 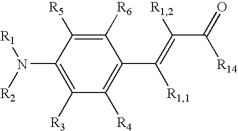
wherein R₁ and R₂ are each independently selected from the group consisting of methyl and alkyl or alternatively, at least one of R₁ or R₂ is part of an aromatic, heteroaromatic, alicyclic or heteroalicyclic ring and where R₁₄ is independently selected from the group consisting of methyl, alkyl, hydroxyalkyl, trihaloalkyl, cycloalkyl, alkenyl, alkynyl, aryl, benzyl, heteroaryl and heteroalicyclic.

31. The method of claim 27, wherein said indicator is a dialkylaminocinnamyl ketone selected from the group consisting of (3E)-4-[4-(dimethylamino)phenyl]-3-buten-2-one, (1E)-1-[4-(dimethylamino)phenyl]-4,4-dimethyl-1-penten-3-one, (2E)-3-[4-(dimethylamino)phenyl]-1-(2-furyl)-2-propen-1-one, (2E)-3-[4-(dimethylamino)phenyl]-1-phenyl-2-propen-1-one, 3-[4-(dimethylamino)phenyl]-1-(4-methylphenyl)-2-propen-1-one, (3E)-4-[4-(diethylamino)phenyl]-3-buten-2-one, 1E)-1-[4-(diethylamino)phenyl]-4,4-dimethyl-1-penten-3-one, (2E)-3-[4-(diethylamino)phenyl]-1-(2-furyl)-2-propen-1-one, (2E)-3-[4-(diethylamino)phenyl]-1-phenyl-2-propen-1-one and 3-[4-(diethylamino)phenyl]-1-(4-methylphenyl)-2-propen-1-one.

32. The method of claim 27, wherein said indicator is a linear p-dialkylaminocinnamic acid of the structure: 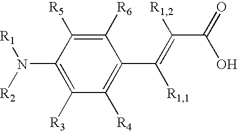
a salt thereof or an ester thereof,
wherein R₁ and R₂ are each independently selected from the group consisting of methyl and alkyl or alternatively, at least one of R₁ or R₂ is part of an aromatic, heteroaromatic, alicyclic or heteroalicyclic ring.

33. The method of claim 27, wherein said indicator is a linear p-dialkylaminocinnamic acid selected from the group consisting of 3-(4-amino-2-methylphenyl)acrylic acid, 4-(dimethylamino)cinnamic acid (CAS 1552-96-1), (2E)-3-[4-(dimethylamino)phenyl]-2-methyl-2-propenoic acid, (2E)-2-cyano-3-[4-(dimethylamino)phenyl]-2-propenoic acid and ethyl 2-cyano-3-[4-(dimethylamino)phenyl]acrylate, salts thereof or esters thereof.

34. The method of claim 27, wherein said indicator is a cyclic p-dialkylaminocinnamic acid (coumarin) of the structure: 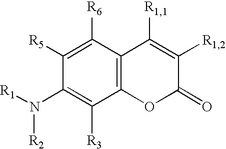
wherein R₁ and R₂ are each independently selected from the group consisting of methyl and alkyl or alternatively, at least one of R₁ or R₂ is part of an aromatic, heteroaromatic, alicyclic or heteroalicyclic ring.

35. The method of claim 27, wherein said indicator is a cyclic p-dialkylaminocinnamic acid (coumarin) selected from the group consisting of coumarin 110 (CAS 20571-42-0), coumarin 6H (CAS 58336-35-9), 3-acetyl-7-(diethylamino)-2H-chromen-2-one, 7-amino-4-methylcoumarin, 7-(diethylamino)coumarin-3,4-dicarboxylic acid (CAS 75240-77-6), 1-[7-(diethylamino)-3-coumarinylcarbonyl]imidazole (CAS 261943-47-9), N-succinimidyl 7-(diethylamino)coumarin-3-carboxylate (CAS 139346-57-9), 7-(diethylamino)coumarin-3-carboxylic acid (CAS 50995-74-9), 7-(diethylamino)coumarin-3-carbonyl azide (CAS 157673-16-0), 7-(diethylamino)coumarin-3-carbohydrazide (CAS 100343-98-4), 7-(diethylamino)coumarin (CAS 20571-42-0), 3-(2-N-methylbenzimidazolyl)-7-N,N-diethylaminocoumarin (CAS 41044-12-6), 3-(2-benzothiazolyl)-7-(diethylamino)coumarin (CAS 38215-36-0), N-succinimidyl 3-(2-benzothiazolyl)-7-(diethylamino)coumarin-4-carboxylate (CAS none) and 3-(2-benzothiazolyl)-7-(diethylamino)coumarin-4-carboxylic acid (CAS 136997-14-3).

36. The method of claim 27, wherein said indicator is a cyclic p-dialkylaminocinnamide (quinolinone) of the structure: 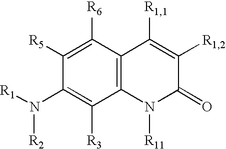
wherein R₁ and R₂ are each independently selected from the group consisting of methyl and alkyl or alternatively, at least one of R₁ or R₂ is part of an aromatic, heteroaromatic, alicyclic or heteroalicyclic ring; and
wherein R₁₁ is selected from the group consisting of H, methyl, alkyl, hydroxyalkyl, trihaloalkyl, cycloalkyl, alkenyl, alkynyl, aryl, benzyl, heteroaryl, heteroalicyclic and halo.

37. The method of claim 27, wherein said indicator is a cyclic p-dialkylaminocinnamamide (quinolinone) selected from the group consisting of 7-(dimethylamino)-4-methyl-2(1H)-quinolinone (CAS 26078-23-9) and 7-(dimethylamino)-2(1H)-quinolinone.

38. The method of claim 27, wherein said indicator is a linear p-dialkylaminocinnamamide of the structure: 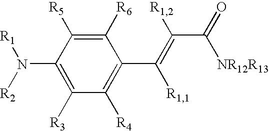
wherein R₁ and R₂ are each independently selected from the group consisting of methyl and alkyl or alternatively, at least one of R₁ or R₂ is part of an aromatic, heteroaromatic, alicyclic or heteroalicyclic ring; and
wherein R₁₂ and R₁₃ are each independently selected from the group consisting of H, methyl, alkyl, hydroxyalkyl, trihaloalkyl, cycloalkyl, alkenyl, alkynyl, aryl, benzyl, heteroaryl, heteroalicyclic and halo.

39. The method of claim 27, wherein said indicator is a linear p-dialkylaminocinnamamide selected from the group consisting of 2-cyano-3-[4-(dimethylamino)phenyl]acrylamide and (2E)-2-cyano-3-[4-(diethylamino)phenyl]-N-methyl-2-propenamide.

40. The method of claim 27, wherein said indicator is a p-dialkylaminobenzaldehyde of the structure: 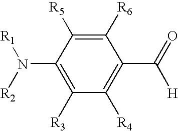
wherein R₁ and R₂ are each independently selected from the group consisting of methyl and alkyl or alternatively, at least one of R₁ or R₂ is part of an aromatic, heteroaromatic, alicyclic or heteroalicyclic ring.

41. The method of claim 27, wherein said indicator is a p-dialkylaminobenzaldehyde selected from the group consisting of p-dimethylaminobenzaldehyde (p-DMAB) and p-diethylaminobenzaldehyde (p-DEAB).

42. The method of claim 27, wherein said indicator is a p-dialkylaminophenyl ketone of the structure: 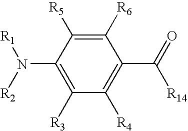
wherein R₁ and R₂ are each independently selected from the group consisting of methyl and alkyl or alternatively, at least one of R₁ or R₂ is part of an aromatic, heteroaromatic, alicyclic or heteroalicyclic ring and where R₁₄ is independently selected from the group consisting of methyl, alkyl, hydroxyalkyl, trihaloalkyl, cycloalkyl, alkenyl, alkynyl, aryl, benzyl, heteroaryl and heteroalicyclic.

43. The method of claim 27, wherein said indicator is a p-dialkylaminophenyl ketone selected from the group consisting of p-dimethylaminophenyl methyl ketone, p-dimethylaminophenyl ethyl ketone, p-dimethylaminophenyl phenyl ketone, p-diethylaminophenyl methyl ketone, p-diethylaminophenyl ethyl ketone, p-diethylaminophenyl phenyl ketone, p-dimethylamino acetophenone 4′-piperidinoacetophenone (CAS 10342-85-5), 4′-piperazinoacetophenone (CAS 51639-48-6), 4′-morpholinoacetophenone (CAS 39910-98-0), 4′-(dimethylamino)-2,2,2-trifluoroacetophenone (CAS 2396-05-6), 1-[4-(4-hydroxy-1-piperidinyl)phenyl]ethanone, 1-[4-(4-morpholinyl)phenyl]-1-propanone and 4-(dimethylamino)phenyl](phenyl)methanone (CAS 530-44-9).

44. The method of claim 27, wherein said indicator is a p-dialkylaminobenzoic acid, of the structure: 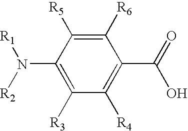
a salt thereof or an ester thereof,
wherein R₁ and R₂ are each are each independently selected from the group consisting of methyl and alkyl or alternatively, at least one of R₁ or R₂ is part of an aromatic, heteroaromatic, alicyclic or heteroalicyclic ring.

45. The method of claim 27, wherein said indicator is a p-dialkylaminobenzoic acid selected from the group consisting of methyl 4-(diphenylamino)benzoate, 4-(dimethylamino)benzoic acid (CAS 619-84-1), 4-(diethylamino)benzoic acid (CAS 5429-28-7), salts thereof or esters thereof.

46. The method of claim 27, wherein said indicator is a p-dialkylaminobenzamide of the structure: 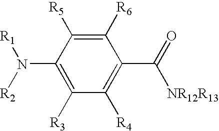
wherein R₁ and R₂ are each independently selected from the group consisting of methyl and alkyl or alternatively, at least one of R₁ or R₂ is part of an aromatic, heteroaromatic, alicyclic or heteroalicyclic ring; and
wherein R₁₂ and R₁₃ are each independently selected from the group consisting of H, methyl, alkyl, hydroxyalkyl, trihaloalkyl, cycloalkyl, alkenyl, alkynyl, aryl, benzyl, heteroaryl, heteroalicyclic and halo.

47. The method of claim 27, wherein said indicator is a p-dialkylaminobenzamide selected from the group consisting of p-dimethylaminobenzamide and p-diethylaminobenzamide.

48. A method for determining the presence or absence of an uronium salt in a sample comprising contacting the sample with an indicator in a indicator solution having a pH greater than about 2, said indicator selected from the group consisting of an aldehyde and a compound configured to form an aromatic dialkylaminium ion upon reaction with the uronium salt.

49. The method of claim 48, wherein said uronium salt is urea nitrate.

50. The method of claim 48, said indicator solution having a pH of greater than about 3.

51. The method of claim 48, said indicator solution having a pH of greater than about 4.

52. The method of claim 48, said indicator solution having a pH of greater than about 5.

53. The method of claim 48, said indicator solution having a pH of greater than about 6.

54. The method of claim 48, said indicator solution having a pH of greater than about 7.

55. The method of claim 48, further comprising allowing a portion of said solution to evaporate so as to increase the concentration of the sample in said indicator solution subsequent to said contacting.

56. The method of claim 48, wherein said indicator solution comprises at least one protic solvent.

57. The method of claim 56, wherein at least one of said at least one protic solvent is selected from the group consisting of water, alcohol, ethanol, methanol, propanol, isopropanol, butanol, isoamylalcohol, glycol and 1,2-dihydroxypropane.

58. The method of claim 56, wherein said indicator solution includes a first protic solvent and at least one second solvent, said second solvent being more volatile than said first protic solvent.

59. The method of claim 58, wherein at least one of said at least one second solvent is selected from the group consisting of ethanol, methanol, butyl acetate, acetonitrile, chloroform, dichloromethane, diethyl ether and ethyl acetate.

60. The method of claim 59, wherein said first protic solvent is water.

61. The method of claim 56, wherein said indicator solution further comprises at least one aprotic solvent.

62. The method of claim 61, wherein at least one of said at least one aprotic solvent is selected from the group consisting of an ether, an acetate, a ketone, acetone, butyl acetate, acetonitrile, chloroform, dichloromethane, diethyl ether, dimethyl formamide, dimethylsulfoxide, ethyl acetate and tetrahydrofuran.

63. The method of claim 56, wherein said solvent is a mixture of ethanol and water.

64. The method of claim 48, wherein said indicator is selected from the group consisting of p-dialkylaminocinnamaldehydes, p-dialkylaminocinnamyl ketones, linear p-dialkylaminocinnamic acids, cyclic p-dialkylaminocinnamic acids (coumarins), linear p-dialkylaminocinnamamides, cyclic p-dialkylaminocinnamamides (quinolinones), p-dialkylaminobenzaldehydes, p-dialkylaminophenyl ketones, p-dialkylaminobenzoic acids, p-dialkylamino benzamides, salts thereof and esters thereof.

65. The method of claim 48, wherein said aldehyde is of the structure R—CHO wherein R is selected from the group consisting of alkyl, hydroxyalkyl, trihaloalkyl, cycloalkyl, alkenyl, alkynyl, aryl, benzyl, heteroaryl, heteroalicyclic, aminobenzyl, alkylaminobenzyl, dialkylaminobenzyl, dialkoxyaminobenzyl, cinnamyl, aminocinnamyl, alkyaminocinnamyl, dialkyaminocinnamyl, alkoxyaminocinnamyl, dialkoxyaminocinnamyl, nitrobenzyl, alkylbenzyl, alkoxybenzyl, phenol, dihydroxyphenyl, trihydroxyphenyl, nitrohydroxyphenyl, hydroxycinnamyl and acetamidophenyl.

66. The method of claim 48, wherein said indicator is an aldehyde selected from the group consisting of o-anisaldehyde, m-anisaldehyde, p-anisaldehyde, 4-acetoxybenzaldehyde, 4-acetamido benzaldehyde, 2-allyloxy benzaldehyde, 4-acetoxy-3,5-dimethoxybenz aldehyde, 4-acetoxy-3-methoxy cinnamaldehyde, α-amyl cinnamaldehyde, 9-anthraldehyde, o-aminobenzoic acid, m-aminobenzoic acid, p-aminobenzoic acid, benzaldehyde, 2-bromobenzaldehyde, 3-bromobenz aldehyde, 4-bromobenz aldehyde, 3-bromo-4-fluorobenz aldehyde, 4-bromo-2-fluorobenz aldehyde, 5-bromosalicyl aldehyde, 2-bromo-3-hydroxy-4-methoxybenz aldehyde, 5-bromo-2-hydroxy-3-methoxybenzaldehyde, 5-bromovanillin, 3-bromo-p-anisaldehyde, 5-bromo-o-anisaldehyde, 5-bromo-3-nitrosalicylaldehyde, 2,5-bis(trifluoromethyl) benzaldehyde, 3,5-bis(trifluoromethyl)benzaldehyde, α-bromocinnamaldehyde, 5-bromo-2,4-dimethoxybenzaldehyde, 5-bromoveratraldehyde, 6-bromoveratraldehyde, benzyloxy acetaldehyde, 4-butoxybenzaldehyde, 3-benzyloxybenzaldehyde, 4-benzyloxy benzaldehyde, 3-(4-methoxyphenoxy)benzaldehyde, 2-benzyloxy-3-methoxy benzaldehyde, 3-benzyloxy-4-methoxybenzaldehyde, 4-benzyloxy-3-methoxy benzaldehyde, 4-biphenylcarboxaldehyde, benzene-1,4-dicarbaldehyde, 2-benzene-1,4-dicarbaldehyde, 2-chlorobenzaldehyde, 3-chlorobenzaldehyde, 4-chlorobenzaldehyde, 2-cyanobenzaldehyde, 3-cyanobenzaldehyde, 4-cyanobenzaldehyde, 2-carboxybenzaldehyde, 3-carboxybenzaldehyde, 4-carboxybenzaldehyde, 2-chloro-4-fluorobenzaldehyde, 2-chloro-6-fluorobenzaldehyde, 3-chloro-4-fluorobenzaldehyde, 2-chloro-5-nitrobenzaldehyde, 2-chloro-6-nitrobenzaldehyde, 4-chloro-3-nitrobenzaldehyde, 5-chlorosalicylaldehyde, 5-chloro-2-nitrobenzaldehyde, cinnamaldehyde, α-chlorocinnamaldehyde, chromone-3-carboxaldehyde, cinnamaldehyde, 4-carboxybenzaldehyde, o-dimethylamino benzaldehyde, o-diethylamino benzaldehyde, o-dimethylamino cinnamaldehyde, o-diethylamino cinnamaldehyde, m-dimethylamino benzaldehyde, m-diethylamino benzaldehyde, m-dimethylamino cinnamaldehyde, m-diethylamino cinnamaldehyde, p-dimethylamino benzaldehyde, p-diethylamino benzaldehyde, p-dimethylamino cinnamaldehyde, p-diethylamino cinnamaldehyde, 2-(difluoromethoxy) benzaldehyde, 4-(difluoromethoxy) benzaldehyde, 2,3-dichlorobenzaldehyde, 2,4-dichlorobenzaldehyde, 2,6-dichlorobenzaldehyde, 3,4-dichlorobenzaldehyde, 3,5-dichlorobenzaldehyde, 2,3-difluorobenzaldehyde, 2,4-difluorobenzaldehyde, 2,5-difluorobenzaldehyde, 2,6-difluorobenzaldehyde, 3,4-difluorobenzaldehyde, 3,5-difluorobenzaldehyde, 2,3-dihydroxybenzaldehyde, 2,4-dihydroxybenzaldehyde, 2,5-dihydroxybenzaldehyde, 3,4-dihydroxybenzaldehyde, 3,5-dihydroxybenzaldehyde, 2,4-dinitrobenzaldehyde, 2,6-dinitrobenzaldehyde, 3,5-dinitrobenzaldehyde, 3,5-dibromosalicylaldehyde, 3,5-dibromo-4-hydroxy benzaldehyde, 3,5-dichloro salicylaldehyde, 3,5-diiodo salicylaldehyde, 3,4-dihydroxy-5-methoxy benzaldehyde, 2,6-dimethoxy-4-hydroxy benzaldehyde, 3,4-dimethoxy-5-hydroxy benzaldehyde, 4,6-dimethoxy salicylaldehyde, 2,3-dimethoxy benzaldehyde, 2,4-dimethoxybenzaldehyde, 2,5-dimethoxybenzaldehyde, 2,6-dimethoxybenzaldehyde, 3,5-dimethoxybenzaldehyde, 2,4-dimethylbenzaldehyde, 2,5-dimethylbenzaldehyde, 3,5-dimethylhydroxylbezaldehyde, 2,3-dimethyl-p-anisaldehyde, 2,5-dimethyl-p-anisaldehyde, 2,4-dimethoxy-3-methylbenzaldehyde, 4-(diethylamino) salicylaldehyde, diphenylacetaldehyde, 3,4-dihydroxybenzaldehyde, 3,5-dimethoxy-4-hydroxy-benzaldehyde, 2,4-dimethoxybenzaldehyde, 2,3-dimethoxybenzaldehyde, 2,5-dimethoxybenzaldehyde, 3,5-dimethoxybenzaldehyde, 3,4-dimethoxybenzaldehyde, 4-dimethylamino-1-naphthaldehyde, 4-dimethylamino-2-methoxybenzaldehyde, 2,4-dihydroxybenzaldehyde, 3,4-dihydroxybenzaldehyde, 2,5-dihydroxybenzaldehyde, 4-dibutylamino-benzaldehyde, 4-diethylamino-2-hydroxybenzaldehyde, 3,5-dimethoxy-4-hydroxy-cinnamaldehyde, 3-methoxy-4-(1-pyrrolidinyl)-benzaldehyde, 4-diethylamino-3-methoxybenzaldehyde, 3,5-dimethyl-4-hydroxybenzaldehyde, 3,5-di-tert-butyl-2-hydroxy benzaldehyde, 3,5-di-tert-butyl-4-hydroxybenzaldehyde, 3,4-dimethoxy-5-hydroxy benzaldehyde, 5-(4-(diethylamino) phenyl)-2,4-pentadienal, 3,4-dihydroxy-benzaldehyde, 3,5-dimethoxy-4-hydroxy-benzaldehyde, 3,5,-dimethyl-4-hydroxybenzaldehyde, 4-dimethylamino-2-methoxy benzaldehyde, 4-dimethylamino-1-naphthaldehyde, 2,3-dimethoxy-benzaldehyde, 2,5-dimethoxybenzaldehyde, 3,5-dimethoxy-benzaldehyde, 3,4-dimethoxy benzaldehyde, 4-dibutylamino-benzaldehyde, 4-diethylamino-2-hydroxy benzaldehyde, 3,4-dimethoxy-5-hydroxy-benzaldehyde, 5-(4-(diethylamino)phenyl)-2,4-pentadienal, 2,4-dihydroxybenzaldehyde, 4-dihydroxy-benzaldehyde, 3,4-dihydroxy-benzaldehyde, 2,5-dihydroxybenzaldehyde, 2,4-dimethoxybenzaldehyde, 3,5-dimethoxy-4-hydroxy-cinnamaldehyde, 4-diethylamino-3-methoxybenzaldehyde, 2-ethoxybenzaldehyde, 4-ethoxybenzaldehyde, 3-ethoxy-4-hydroxybenzaldehyde, 3-ethoxysalicylaldehyde, 3-ethoxy-4-hydroxy benzaldehyde, N-ethylcarbazole-3-aldehyde, 3-ethoxy-4-methoxy benzaldehyde, 4-ethoxy benzaldehyde, 4-ethoxy benzaldehyde, 3-ethoxy-4-hydroxy benzaldehyde, 2-fluoro benzaldehyde, 3-fluorobenzaldehyde, 4-fluorobenzaldehyde, 2-formylphenylboronic acid, 3-formylphenylboronic acid, 4-formylphenylboronic acid, 2-formylbenzenesulfonic acid, 2-fluoro-5-nitrobenzaldehyde, 3-fluorosalicylaldehyde, 4-formyl-1,3-benzenedisulfonic acid, 2-fluoro-3-(trifluoromethyl)benzaldehyde, 2-fluoro-6-(trifluoromethyl)benzaldehyde, 4-fluoro-2-(trifluoromethyl) benzaldehyde, 4-fluoro-3-(trifluoromethyl)benzaldehyde, 3-fluoro-p-anisaldehyde, 3-fluoro-2-methlbenzaldehyde, 4-ethylbenzaldehyde, 2-fluorenecarboxaldehyde, 3-hydroxybenzaldehyde, 4-hydroxy benzaldehyde, 2-hydroxy-5-nitrobenzaldehyde, 3-hydroxy-4-nitro benzaldehyde, 4-hydroxy-3-nitro benzaldehyde, 5-hydroxy-2-nitrobenzaldehyde, 2-hydroxy-4-methoxybenzaldehyde, 2-hydroxy-5-methoxybenzaldehyde, 3-hydroxy-4-methoxybenzaldehyde, 2-hydroxy-5-methyl-1,3-benzenedicarboxaldehyde, hydrocinnamaldehyde, 2-hydroxy-1-naphthaldehyde, 4-(hexyloxy)benzaldehyde, 4-hydroxy-3-methoxybenzaldehyde (vanillin), 3-hydroxy-4-methoxy-benzaldehyde (isovanillin), 4-hydroxybenzaldehyde, 4-hydroxy-2-methoxybenzaldehyde, 2-hydroxybenzaldehyde, 4-hydroxy-1-naphthaldehyde, 4-methoxy-1-naphthaldehyde, 4′-hydroxy-biphenyl-1-carbaldehyde, 2-hydroxy-3-methoxybenzaldehyde, 4-hydroxy-3-methoxy-cinnamaldehyde, 6-hydroxychromen-3-carbox-aldehyde, α-hexylcinnamaldehyde, 4-hydroxy-3-methoxy-cinnamaldehyde, 4-hydroxy-benzaldehyde, 4-hydroxy-2-methoxy-benzaldehyde, 2-hydroxybenzaldehyde, 4-hydroxy-1-naphthaldehyde, 4′-hydroxy-biphenyl-1-carbaldehyde, 2-hydroxy-3-methoxybenzaldehyde, isophthalaldehyde, 5-iodovanillin, 4-isopropylbenzaldehyde, indole-3-carbaldehyde, 6-methyl-2-pyridinecarboxaldehyde, 2,3-(methylenedioxy) benzaldehyde, 3-methoxy-5-nitrosalicylaldehyde, 3-methyl-p-anisaldehyde, 2-methoxycinnamaldehyde, mesitaldehyde, 2-methoxy-1-naphthaldehyde, N-methylpyrrole-2-aldehyde, 5-methylfurfural, 6-methylindole-3-carboxaldehyde, 6-methyl-4-oxo-1 (4H)-benzopyran-3-carbaldehyde, 2-methyl-1,4-naphthoquinone, 4-carboxybenzaldehyde, 4-methoxy-1-naphthalaldehyde, methyl 2-formyl-3,5-dimethoxybenzoate, 2-methoxy-1-naphthalaldehyde, 4-methyl-5-imidazole-carboxaldehyde, 2-methyl-1,4-naphthoquinone, 3-methoxy-4-(1-pyrrolidinyl)-benzaldehyde, 2-methoxy-1-naphthaldehyde, 4-methoxy-1-naphthaldehyde, 2-nitrobenzaldehyde, 3-nitrobenzaldehyde, 4-nitrobenzaldehyde, 5-nitro-vanillin, 2-nitrocinnamaldehyde, 4-nitrocinnamaldehyde, 2-nitrobenzaldehyde, 3-nitrobenzaldehyde, 4-nitrobenzaldehyde, 6-nitroveratraldehyde, 1-naphthaldehyde, 2-naphthaldehyde, 2,3-naphthalendicarboxaldehyde, 1,8-naphthalaldehydic acid, 2-nitrobenzaldehyde, 3-nitrobenzaldehyde, 4-nitrobenzaldehyde, phenylacetaldehyde, 2-pyridinecarboxaldehyde, 3-pyridinecarboxaldehyde, 4-pyridinecarboxaldehyde, 4-pyridinecarboxaldehyde N-oxide, phthalaldehyde, piperonal, 2-phenyl propionaldehyde, pentafluorobenzaldehyde, 3-phenylbutyraldehyde, 4-propoxybenzaldehyde, pentamethylbenzaldehyde, 3-phenoxybenzaldehyde, 4-phenoxybenzaldehyde, phenylpropargyl aldehyde, 1,2-phthaldialdehyde, pyrrole-2-aldehyde, phthalimidoacetaldehyde, o-phtalaldehyde, 1,2-phthaldialdehyde, 2-quinolinecarboxaldehyde, 3-quinolinecarboxaldehyde, 4-quinolinecarboxaldehyde, salicylaldehyde, syringaldeyde, o-tolualdehyde, m-tolualdehyde, p-tolualdehyde, 3-(trifluoromethoxy)benzaldehyde, 4-(trifluoromethoxy)benzaldehyde α,α,α-trifluoro-o-tolualdehyde α,α,α-trifluoro-m-tolualdehyde, α,α,α-trifluoro-p-tolualdehyde, terephthalaldehyde, 5-(trifluoromethoxy)salicylaldehyde, 2,3,5-trichloro benzaldehyde, 2,3,6-trichlorobenzaldehyde, 2,3,4-trifluorobenzaldehyde, 2,3,6-trifluoro benzaldehyde, 2,3,4-trihydroxy benzaldehyde, 2,4,6-trihydroxybenzaldehyde, 3,4,5-trihydroxy benzaldehyde, 2,3,5,6-tetrafluorobenzaldehyde, 2,3,4-trimethoxy benzaldehyde, 2,4,5-trimethoxybenzaldehyde, 2,4,6-trimethoxybenzaldehyde, 3,4,5-trimethoxy benzaldehyde, 4-4-tert-butylbenzaldehyde, 3-tert-butyl-2-hydroxy benzaldehyde, 5-tert-butyl-2-hydroxy benzaldehyde, 2,3,4-trihydroxy benzaldehyde, 3,4,5-trihydroxybenzaldehyde, 2,3-thiophene-dicarboxaldehyde, 2,5-thiophene-dicarboxaldehyde, thiophene-2-aldehyde, thiophene-3-aldehyde, 2,4,6-trihydroxy benzaldehyde, 2,3,4-trihydroxybenzaldehyde, 3,4,5-trihydroxybenzaldehyde, 2,4,6-trihydroxy benzaldehyde, vanillin, o-vanillin, vinyl benzaldehyde, veratraldehyde and vanillin acetate.

67. The method of claim 48, wherein said aldehyde is an aromatic aldehyde of the structure Ar—(CR_{n,a}═CR_{n,b})ₙ—CH═O where:
n is an integer 0 or greater;
Ar is aromatic; and
each one of R_{n,a} and R_{n,b} are independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl, cyano, nitro, amino, —NR₁₅R₁₆, wherein R₁₅ and R₁₆ are independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl and cyano, or, alternatively, at least two of substituents of Ar, R₁₅, R₁₆, R_{n,a} and/or R_{n,b} form an aromatic, heteroaromatic, alicyclic or heteroalicyclic ring.

68. The method of claim 67, wherein said aromatic group Ar is substituted with at least one positive charge stabilizing functional group.

69. The method of claim 68, wherein at least one of said at least one positive charge stabilizing functional groups is para to said —(CR_{n,a}═CR_{n,b})ₙ—CH═O group.

70. The method of claim 68, wherein at least one of said at least one positive charge stabilizing functional groups is selected from the group consisting of amines, alkylamines, dialkylamines, alcohols, esters, amides, acids and alkyls.

71. The method of claim 68, wherein at least one of said at least one positive charge stabilizing functional groups is a disubstituted amine.

72. The method of claim 71, wherein said disubstituted amine is a dialkyl amine.

73. The method of claim 72, wherein said aromatic aldehyde is selected from the group consisting of p-DMAC, p-DMAB, p-DEAC and p-DEAB.

74. The method of claim 48, wherein the concentration of said indicator in said indicator solution is less than about 4% by weight.

75. The method of claim 74, wherein the concentration of said indicator in said indicator solution is less than about 1% by weight.

76. The method of claim 74, wherein the concentration of said indicator in said indicator solution is less than about 0.5% by weight.

77. The method of claim 48, wherein the concentration of said indicator in said indicator solution is greater than about 0.1% by weight.

78. The method of claim 48, further comprising collecting and/or concentrating the sample.

79. The method of claim 78, wherein said collecting and/or said concentrating is performed prior to said contacting.

80. The method of claim 78, wherein said collecting and/or said concentrating comprises gathering the sample from an area.

81. The method of claim 80, wherein said gathering the sample includes wiping said area with a collector and/or a concentrator.

82. The method of claim 81, wherein said collector and/or said concentrator is selected from the group consisting of consisting of bibulous materials, cloth, fabrics, felt, flannel, membranes, pads, papers, sponges, swabs, swatches, tissues and wipes.

83. The method of claim 81, wherein said contacting occurs on said collector and/or said concentrator.

84. The method of claim 83, wherein said collector and/or said concentrator is impregnated with said indicator solution prior to said wiping.

85. The method of claim 83, further comprising dispensing said indicator onto said collector and/or said concentrator prior to said wiping.

86. The method of claim 83, further comprising dispensing said indicator onto said collector and/or said concentrator subsequent to said wiping.

87. The method of claim 48, wherein said contacting occurs by applying said indicator solution to a location where the sample is found.

88. The method of claim 85, wherein said applying includes dispensing said indicator from a vessel selected from the group consisting of aerosol dispensers, bags, beakers, bottles, droppers, jars, mechanically actuated spray dispensers, pressurized spray dispensers, pump spray dispensers, sacks, sachet, spray dispensers, squeeze bottles and tube.

89. The method of claim 48, further comprising observing a change in said indicator solution subsequent to said contacting.

90. The method of claim 89, wherein said change in said indicator solution is selected from the group of a change in color, a change in light absorption and a change in fluorescence.

91. A kit for determining the presence or absence of an uronium salt in a sample comprising:
a) at least one indicator selected from the group consisting of an aldehyde and a compound configured to form an aromatic dialkylaminium ion upon reaction with the uronium salt;
b) a device selected from amongst a collector, a concentrator or a combination thereof; and
c) a vessel configured to hold and dispense a solution of said indicator having a pH of greater than about 2.

92. The kit of claim 91, wherein said uronium salt is urea nitrate.

93. The kit of claim 91, said indicator packaged in a packaging material and identified in print, in or on said packaging material, for use for determining the presence or absence of an uronium salt in a sample.

94. A kit for determining the presence or absence of an explosive in a sample comprising:
a) at least one indicator selected from the group consisting of an aldehyde and a compound configured to form an aromatic dialkylaminium ion upon reaction with an uronium salt;
b) a device selected from amongst a collector, a concentrator or a combination thereof; and
c) a vessel configured to hold and dispense a solution of said indicator having a pH of greater than about 2.

95. The kit of claim 91, wherein said explosive comprises urea nitrate.

96. The kit of claim 94, said indicator packaged in a packaging material and identified in print, in or on said packaging material, for use for determining the presence or absence of an explosive in a sample.

97. The kit of claim 91, wherein at least one said indicator is provided in a concentrated form.

98. The kit of claim 97, wherein at least one said indicator is provided as a substantially pure solid.

99. The kit of claim 97, wherein at least one said indicator is provided as a substantially pure liquid.

100. The kit of claim 97, wherein at least one said indicator is provided in a concentrated form comprising greater than about 1% by weight of said indicator.

101. The kit of claim 97 further comprising a solvent, said solvent chosen so that upon mixing with said concentrated form of said indicator, an indicator solution having a pH of greater than about 2 is made.

102. The kit of claim 91, wherein said at least one indicator is provided in an indicator solution comprising a solvent and having a pH of greater than about 2.

103. The kit of claim 102, wherein the concentration of said indicator in said indicator solution is less than about 4% by weight.

104. The kit of claim 102, wherein the concentration of said indicator in said indicator solution is less than about 1% by weight.

105. The kit of claim 102, wherein the concentration of said indicator in said indicator solution is less than about 0.5% by weight.

106. The kit of claim 102, wherein the concentration of said indicator in said indicator solution is greater than about 0.1% by weight.

107. The kit of claim 101, wherein said pH is greater than about 3.

108. The kit of claim 101, wherein said pH is greater than about 4.

109. The kit of claim 101, wherein said pH is greater than about 5.

110. The kit of claim 101, wherein said pH is greater than about 6.

111. The kit of claim 101, wherein said pH is greater than about 7.

112. The of claim 101, wherein said solvent comprises at least one protic solvent.

113. The kit of claim 101, wherein at least one of said at least one protic solvents is selected from the group consisting of water, alcohol, ethanol, methanol, propanol, isopropanol, butanol, isoamylalcohol, glycol and 1,2-dihydroxypropane.

114. The kit of claim 101, wherein said solvent comprises, in addition to a first protic solvent, a second solvent, said second solvent being more volatile than said first protic solvent.

115. The kit of claim 114, wherein at least one of said at least one second solvent is selected from the group consisting of ethanol, methanol, butyl acetate, acetonitrile, chloroform, dichloromethane, diethyl ether and ethyl acetate.

116. The kit of claim 114, wherein said first protic solvent is water.

117. The kit of claim 101, wherein said solvent further comprises at least one aprotic solvent.

118. The kit of claim 101, wherein at least one of said at least one aprotic solvent is selected from the group consisting of an ether, an acetate, a ketone, acetone, butyl acetate, acetonitrile, chloroform, dichloromethane, diethyl ether, dimethyl formamide, dimethyl sulfoxide, ethyl acetate and tetrahydrofuran.

119. The kit of claim 101, wherein said solvent is a mixture of ethanol with water.

120. The kit of claim 91, wherein said collector and/or said concentrator is selected from the group consisting of bibulous materials, cloth, fabrics, felt, flannel, membranes, pads, papers, sponges, swabs, swatches, tissues and wipes.

121. The kit of claim 97, wherein said collector and/or said concentrator is impregnated with said indicator.

122. The kit of claim 91, wherein said vessel is selected from the group consisting of aerosol dispensers, bags, beakers, bottles, droppers, jars, mechanically actuated spray dispensers, pressurized spray dispensers, pump spray dispensers, sacks, sachet, spray dispensers, squeeze bottles, ampoules, syringes, capsules, foil wrappers and tubes.

123. The kit of claim 91, wherein said indicator is selected from the group consisting of p-dialkylaminocinnamaldehydes, p-dialkylaminocinnamyl ketones, linear p-dialkylaminocinnamic acids, cyclic p-dialkylaminocinnamic acids (coumarins), linear p-dialkylaminocinnamamides, cyclic p-dialkylaminocinnamamides (quinolinones), p-dialkylaminobenzaldehydes, p-dialkylaminophenyl ketones, p-dialkylaminobenzoic acids, p-dialkylamino benzamides, salts thereof and esters thereof.

124. The kit of claim 91, wherein said aldehyde is of the structure R—CHO wherein R is selected from the group consisting of alkyl, hydroxyalkyl, trihaloalkyl, cycloalkyl, alkenyl, alkynyl, aryl, benzyl, heteroaryl, heteroalicyclic, aminobenzyl, alkylaminobenzyl, dialkylaminobenzyl, dialkoxyaminobenzyl, cinnamyl, aminocinnamyl, alkyaminocinnamyl, dialkyaminocinnamyl, alkoxyaminocinnamyl, dialkoxyaminocinnamyl, nitrobenzyl, alkylbenzyl, alkoxybenzyl, phenol, dihydroxyphenyl, trihydroxyphenyl, nitrohydroxyphenyl, hydroxycinnamyl and acetamidophenyl.

125. The kit of claim 91, wherein said indicator is an aldehyde selected from the group consisting of o-anisaldehyde, m-anisaldehyde, p-anisaldehyde, 4-acetoxybenzaldehyde, 4-acetamido benzaldehyde, 2-allyloxy benzaldehyde, 4-acetoxy-3,5-dimethoxybenz aldehyde, 4-acetoxy-3-methoxy cinnamaldehyde, α-amyl cinnamaldehyde, 9-anthraldehyde, o-aminobenzoic acid, m-aminobenzoic acid, p-aminobenzoic acid, benzaldehyde, 2-bromobenzaldehyde, 3-bromobenz aldehyde, 4-bromobenz aldehyde, 3-bromo-4-fluorobenz aldehyde, 4-bromo-2-fluorobenz aldehyde, 5-bromosalicyl aldehyde, 2-bromo-3-hydroxy-4-methoxybenz aldehyde, 5-bromo-2-hydroxy-3-methoxybenzaldehyde, 5-bromovanillin, 3-bromo-p-anisaldehyde, 5-bromo-o-anisaldehyde, 5-bromo-3-nitrosalicylaldehyde, 2,5-bis(trifluoromethyl) benzaldehyde, 3,5-bis(trifluoromethyl)benzaldehyde, α-bromocinnamaldehyde, 5-bromo-2,4-dimethoxybenzaldehyde, 5-bromoveratraldehyde, 6-bromoveratraldehyde, benzyloxy acetaldehyde, 4-butoxybenzaldehyde, 3-benzyloxybenzaldehyde, 4-benzyloxy benzaldehyde, 3-(4-methoxyphenoxy)benzaldehyde, 2-benzyloxy-3-methoxy benzaldehyde, 3-benzyloxy-4-methoxybenzaldehyde, 4-benzyloxy-3-methoxy benzaldehyde, 4-biphenylcarboxaldehyde, benzene-1,4-dicarbaldehyde, 2-benzene-1,4-dicarbaldehyde, 2-chlorobenzaldehyde, 3-chlorobenzaldehyde, 4-chlorobenzaldehyde, 2-cyanobenzaldehyde, 3-cyanobenzaldehyde, 4-cyanobenzaldehyde, 2-carboxybenzaldehyde, 3-carboxybenzaldehyde, 4-carboxybenzaldehyde, 2-chloro-4-fluorobenzaldehyde, 2-chloro-6-fluorobenzaldehyde, 3-chloro-4-fluorobenzaldehyde, 2-chloro-5-nitrobenzaldehyde, 2-chloro-6-nitrobenzaldehyde, 4-chloro-3-nitrobenzaldehyde, 5-chlorosalicylaldehyde, 5-chloro-2-nitrobenzaldehyde, cinnamaldehyde, α-chlorocinnamaldehyde, chromone-3-carboxaldehyde, cinnamaldehyde, 4-carboxybenzaldehyde, o-dimethylamino benzaldehyde, o-diethylamino benzaldehyde, o-dimethylamino cinnamaldehyde, o-diethylamino cinnamaldehyde, m-dimethylamino benzaldehyde, m-diethylamino benzaldehyde, m-dimethylamino cinnamaldehyde, m-diethylamino cinnamaldehyde, p-dimethylamino benzaldehyde, p-diethylamino benzaldehyde, p-dimethylamino cinnamaldehyde, p-diethylamino cinnamaldehyde, 2-(difluoromethoxy) benzaldehyde, 4-(difluoromethoxy) benzaldehyde, 2,3-dichlorobenzaldehyde, 2,4-dichlorobenzaldehyde, 2,6-dichlorobenzaldehyde, 3,4-dichlorobenzaldehyde, 3,5-dichlorobenzaldehyde, 2,3-difluorobenzaldehyde, 2,4-difluorobenzaldehyde, 2,5-difluorobenzaldehyde, 2,6-difluorobenzaldehyde, 3,4-difluorobenzaldehyde, 3,5-difluorobenzaldehyde, 2,3-dihydroxybenzaldehyde, 2,4-dihydroxybenzaldehyde, 2,5-dihydroxybenzaldehyde, 3,4-dihydroxybenzaldehyde, 3,5-dihydroxybenzaldehyde, 2,4-dinitrobenzaldehyde, 2,6-dinitrobenzaldehyde, 3,5-dinitrobenzaldehyde, 3,5-dibromosalicylaldehyde, 3,5-dibromo-4-hydroxy benzaldehyde, 3,5-dichloro salicylaldehyde, 3,5-diiodo salicylaldehyde, 3,4-dihydroxy-5-methoxy benzaldehyde, 2,6-dimethoxy-4-hydroxy benzaldehyde, 3,4-dimethoxy-5-hydroxy benzaldehyde, 4,6-dimethoxy salicylaldehyde, 2,3-dimethoxy benzaldehyde, 2,4-dimethoxybenzaldehyde, 2,5-dimethoxybenzaldehyde, 2,6-dimethoxybenzaldehyde, 3,5-dimethoxybenzaldehyde, 2,4-dimethylbenzaldehyde, 2,5-dimethylbenzaldehyde, 3,5-dimethylhydroxylbezaldehyde, 2,3-dimethyl-p-anisaldehyde, 2,5-dimethyl-p-anisaldehyde, 2,4-dimethoxy-3-methylbenzaldehyde, 4-(diethylamino) salicylaldehyde, diphenylacetaldehyde, 3,4-dihydroxybenzaldehyde, 3,5-dimethoxy-4-hydroxy-benzaldehyde, 2,4-dimethoxybenzaldehyde, 2,3-dimethoxybenzaldehyde, 2,5-dimethoxybenzaldehyde, 3,5-dimethoxybenzaldehyde, 3,4-dimethoxybenzaldehyde, 4-dimethylamino-1-naphthaldehyde, 4-dimethylamino-2-methoxybenzaldehyde, 2,4-dihydroxybenzaldehyde, 3,4-dihydroxybenzaldehyde, 2,5-dihydroxybenzaldehyde, 4-dibutylamino-benzaldehyde, 4-diethylamino-2-hydroxybenzaldehyde, 3,5-dimethoxy-4-hydroxy-cinnamaldehyde, 3-methoxy-4-(1-pyrrolidinyl)-benzaldehyde, 4-diethylamino-3-methoxybenzaldehyde, 3,5-dimethyl-4-hydroxybenzaldehyde, 3,5-di-tert-butyl-2-hydroxy benzaldehyde, 3,5-di-tert-butyl-4-hydroxybenzaldehyde, 3,4-dimethoxy-5-hydroxy benzaldehyde, 5-(4-(diethylamino) phenyl)-2,4-pentadienal, 3,4-dihydroxy-benzaldehyde, 3,5-dimethoxy-4-hydroxy-benzaldehyde, 3,5,-dimethyl-4-hydroxybenzaldehyde, 4-dimethylamino-2-methoxy benzaldehyde, 4-dimethylamino-1-naphthaldehyde, 2,3-dimethoxy-benzaldehyde, 2,5-dimethoxybenzaldehyde, 3,5-dimethoxy-benzaldehyde, 3,4-dimethoxy benzaldehyde, 4-dibutylamino-benzaldehyde, 4-diethylamino-2-hydroxy benzaldehyde, 3,4-dimethoxy-5-hydroxy-benzaldehyde, 5-(4-(diethylamino)phenyl)-2,4-pentadienal, 2,4-dihydroxybenzaldehyde, 4-dihydroxy-benzaldehyde, 3,4-dihydroxy-benzaldehyde, 2,5-dihydroxybenzaldehyde, 2,4-dimethoxybenzaldehyde, 3,5-dimethoxy-4-hydroxy-cinnamaldehyde, 4-diethylamino-3-methoxybenzaldehyde, 2-ethoxybenzaldehyde, 4-ethoxybenzaldehyde, 3-ethoxy-4-hydroxybenzaldehyde, 3-ethoxysalicylaldehyde, 3-ethoxy-4-hydroxy benzaldehyde, N-ethylcarbazole-3-aldehyde, 3-ethoxy-4-methoxy benzaldehyde, 4-ethoxy benzaldehyde, 4-ethoxy benzaldehyde, 3-ethoxy-4-hydroxy benzaldehyde, 2-fluoro benzaldehyde, 3-fluorobenzaldehyde, 4-fluorobenzaldehyde, 2-formylphenylboronic acid, 3-formylphenylboronic acid, 4-formylphenylboronic acid, 2-formylbenzenesulfonic acid, 2-fluoro-5-nitrobenzaldehyde, 3-fluorosalicylaldehyde, 4-formyl-1,3-benzenedisulfonic acid, 2-fluoro-3-(trifluoromethyl)benzaldehyde, 2-fluoro-6-(trifluoromethyl)benzaldehyde, 4-fluoro-2-(trifluoromethyl) benzaldehyde, 4-fluoro-3-(trifluoromethyl)benzaldehyde, 3-fluoro-p-anisaldehyde, 3-fluoro-2-methlbenzaldehyde, 4-ethylbenzaldehyde, 2-fluorenecarboxaldehyde, 3-hydroxybenzaldehyde, 4-hydroxy benzaldehyde, 2-hydroxy-5-nitrobenzaldehyde, 3-hydroxy-4-nitro benzaldehyde, 4-hydroxy-3-nitro benzaldehyde, 5-hydroxy-2-nitrobenzaldehyde, 2-hydroxy-4-methoxybenzaldehyde, 2-hydroxy-5-methoxybenzaldehyde, 3-hydroxy-4-methoxybenzaldehyde, 2-hydroxy-5-methyl-1,3-benzenedicarboxaldehyde, hydrocinnamaldehyde, 2-hydroxy-1-naphthaldehyde, 4-(hexyloxy)benzaldehyde, 4-hydroxy-3-methoxybenzaldehyde (vanillin), 3-hydroxy-4-methoxy-benzaldehyde (isovanillin), 4-hydroxybenzaldehyde, 4-hydroxy-2-methoxybenzaldehyde, 2-hydroxybenzaldehyde, 4-hydroxy-1-naphthaldehyde, 4-methoxy-1-naphthaldehyde, 4′-hydroxy-biphenyl-1-carbaldehyde, 2-hydroxy-3-methoxybenzaldehyde, 4-hydroxy-3-methoxy-cinnamaldehyde, 6-hydroxychromen-3-carbox-aldehyde, α-hexylcinnamaldehyde, 4-hydroxy-3-methoxy-cinnamaldehyde, 4-hydroxy-benzaldehyde, 4-hydroxy-2-methoxy-benzaldehyde, 2-hydroxybenzaldehyde, 4-hydroxy-1-naphthaldehyde, 4′-hydroxy-biphenyl-1-carbaldehyde, 2-hydroxy-3-methoxybenzaldehyde, isophthalaldehyde, 5-iodovanillin, 4-isopropylbenzaldehyde, indole-3-carbaldehyde, 6-methyl-2-pyridinecarboxaldehyde, 2,3-(methylenedioxy) benzaldehyde, 3-methoxy-5-nitrosalicylaldehyde, 3-methyl-p-anisaldehyde, 2-methoxycinnamaldehyde, mesitaldehyde, 2-methoxy-1-naphthaldehyde, N-methylpyrrole-2-aldehyde, 5-methylfurfural, 6-methylindole-3-carboxaldehyde, 6-methyl-4-oxo-1 (4H)-benzopyran-3-carbaldehyde, 2-methyl-1,4-naphthoquinone, 4-carboxybenzaldehyde, 4-methoxy-1-naphthalaldehyde, methyl 2-formyl-3,5-dimethoxybenzoate, 2-methoxy-1-naphthalaldehyde, 4-methyl-5-imidazole-carboxaldehyde, 2-methyl-1,4-naphthoquinone, 3-methoxy-4-(1-pyrrolidinyl)-benzaldehyde, 2-methoxy-1-naphthaldehyde, 4-methoxy-1-naphthaldehyde, 2-nitrobenzaldehyde, 3-nitrobenzaldehyde, 4-nitrobenzaldehyde, 5-nitro-vanillin, 2-nitrocinnamaldehyde, 4-nitrocinnamaldehyde, 2-nitrobenzaldehyde, 3-nitrobenzaldehyde, 4-nitrobenzaldehyde, 6-nitroveratraldehyde, 1-naphthaldehyde, 2-naphthaldehyde, 2,3-naphthalendicarboxaldehyde, 1,8-naphthalaldehydic acid, 2-nitrobenzaldehyde, 3-nitrobenzaldehyde, 4-nitrobenzaldehyde, phenylacetaldehyde, 2-pyridinecarboxaldehyde, 3-pyridinecarboxaldehyde, 4-pyridinecarboxaldehyde, 4-pyridinecarboxaldehyde N-oxide, phthalaldehyde, piperonal, 2-phenyl propionaldehyde, pentafluorobenzaldehyde, 3-phenylbutyraldehyde, 4-propoxybenzaldehyde, pentamethylbenzaldehyde, 3-phenoxybenzaldehyde, 4-phenoxybenzaldehyde, phenylpropargyl aldehyde, 1,2-phthaldialdehyde, pyrrole-2-aldehyde, phthalimidoacetaldehyde, o-phtalaldehyde, 1,2-phthaldialdehyde, 2-quinolinecarboxaldehyde, 3-quinolinecarboxaldehyde, 4-quinolinecarboxaldehyde, salicylaldehyde, syringaldeyde, o-tolualdehyde, m-tolualdehyde, p-tolualdehyde, 3-(trifluoromethoxy)benzaldehyde, 4-(trifluoromethoxy)benzaldehyde α,α,α-trifluoro-o-tolualdehyde α,α,α-trifluoro-m-tolualdehyde, α,α,α-trifluoro-p-tolualdehyde, terephthalaldehyde, 5-(trifluoromethoxy)salicylaldehyde, 2,3,5-trichloro benzaldehyde, 2,3,6-trichlorobenzaldehyde, 2,3,4-trifluorobenzaldehyde, 2,3,6-trifluoro benzaldehyde, 2,3,4-trihydroxy benzaldehyde, 2,4,6-trihydroxybenzaldehyde, 3,4,5-trihydroxy benzaldehyde, 2,3,5,6-tetrafluorobenzaldehyde, 2,3,4-trimethoxy benzaldehyde, 2,4,5-trimethoxybenzaldehyde, 2,4,6-trimethoxybenzaldehyde, 3,4,5-trimethoxy benzaldehyde, 4-4-tert-butylbenzaldehyde, 3-tert-butyl-2-hydroxy benzaldehyde, 5-tert-butyl-2-hydroxy benzaldehyde, 2,3,4-trihydroxy benzaldehyde, 3,4,5-trihydroxybenzaldehyde, 2,3-thiophene-dicarboxaldehyde, 2,5-thiophene-dicarboxaldehyde, thiophene-2-aldehyde, thiophene-3-aldehyde, 2,4,6-trihydroxy benzaldehyde, 2,3,4-trihydroxybenzaldehyde, 3,4,5-trihydroxybenzaldehyde, 2,4,6-trihydroxy benzaldehyde, vanillin, o-vanillin, vinyl benzaldehyde, veratraldehyde and vanillin acetate.

126. The kit of claim 91, wherein said aldehyde is an aromatic aldehyde of the structure Ar—(CR_{n,a}═CR_{n,b})ₙ—CH═O where:
n is an integer 0 or greater;
Ar is aromatic; and
each one of R_{n,a} and R_{n,b} are independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl, cyano, nitro, amino, —NR₁₅R₁₆, where R₁₅ and R₁₆ are independently selected from the group consisting of H, methyl, alkyl, hydroxyalkyl, trihaloalkyl, cycloalkyl, alkenyl, alkynyl, aryl, benzyl, heteroaryl, heteroalicyclic, halo, hydroxy, alkoxy, aryloxy, thiohydroxy, thioalkoxy, thioaryloxy, sulfinyl, sulfonyl and cyano, or, alternatively, at least two of substituents of Ar, R₁₅, R₁₆, R_{n,a} and/or R_{n,b} form an aromatic, heteroaromatic, alicyclic or heteroalicyclic ring.

127. The kit of claim 126, wherein said aromatic group Ar is substituted with at least one positive charge stabilizing functional group.

128. The kit of claim 127, wherein at least one of said at least one positive charge stabilizing functional groups is para to said —(CR_{n,a}═CR_{n,b})ₙ—CH═O group.

129. The kit of claim 127, wherein at least one of said at least one positive charge stabilizing functional groups is selected from the group consisting of amines, alkylamines, dialkylamines, alcohols, esters, amides, acids and alkyls.

130. The kit of claim 127, wherein at least one of said at least one positive charge stabilizing functional groups is a disubstituted amine.

131. The kit of claim 130, wherein said disubstituted amine is a dialkyl amine.

132. The kit of claim 131, wherein said aromatic aldehyde is selected from the group consisting of p-DMAC, p-DMAB, p-DEAC and p-DEAB.
